(19) United States
(12) Patent Application Publication
(10) Pub. No.: US 2026/0305953 A1
(43) Pub. Date: Oct. 8, 2026

(54) IMPLANTABLE PRESSURE SENSOR SYSTEM FOR MEASUREMENT AND WIRELESS TRANSMISSION OF INTERNAL PRESSURES

(71) Applicant: Qura, Inc., Duxbury, MA (US)

(72) Inventors: William G. Hendren, Duxbury, MA (US); Stefan Bauer, Bern (CH); Jean-Noel Fehr, Neuchatel (CH)

(73) Assignee: Qura, Inc., Duxbury, MA (US)

(21) Appl. No.: 19/483,000

(22) PCT Filed: May 10, 2024
(86) PCT No.: PCT/US2024/028861

§ 371 Date: Nov. 10, 2025

Related U.S. Application Data
(60) Provisional application No. 63/501,193, filed on May 10, 2023.

(51) Int. Cl. A61B 5/0205; A61B 3/16; A61B 5/00; A61B 5/0215; A61B 5/03; A61B 5/145

(52) U.S. Cl. CPC A61B 5/0205; A61B 3/16; A61B 5/0215; A61B 5/03; A61B 5/14503; A61B 5/14542; A61B 5/6821; A61B 5/686; A61B 5/6862; A61B 5/7271; A61B 2560/0214; A61B 2562/0247; A61B 2562/168; A61B 2562/227

(57) ABSTRACT

Implantable pressure-sensing systems where the sensing and electronic functions are anatomically distributed in different locations for reduced size and reduced risk of complications are disclosed. An inventive implantable pressure-sensing device can include a pressure-sensing element, readout electronics, and a bridge connecting and operably coupling the pressure-sensing element to the readout electronics. The implantable pressure system(s) can include a conformal coating that hermetically seals the system's modules with biocompatible surfaces before being implanted. An inventive system can be used in a wide range of applications for implanted pressure sensing, including intraocular, cardiovascular, and bladder applications.

### CROSS REFERENCE TO RELATED APPLICATION(S)

The present application claims the priority benefit, under 35 U.S.C. 119(e), of U.S. Application No. 63/501,193, filed May 10, 2023, which is incorporated herein by reference in its entirety for all purposes.

### BACKGROUND

Pressure in various organs of the body (heart and blood vessels, eye, bladder, and brain) is highly regulated. Measurement of these pressures can provide an indicator of patient health or progression of disease. The management of pressure-mediated chronic disease such as glaucoma, hypertension, heart failure, and many others is significantly limited by the ability to obtain accurate and continuous measurement of relevant pressures in an “out of hospital” or outpatient setting. While non-invasive methods for measuring pressure have been explored extensively, these methods do not provide adequate precision and accuracy. Simultaneously, non-invasive methods are heavily dependent on patient interaction, and as such are subject to patient non-compliance.

Measurements of pressure are routinely used in a wide range of disciplines in medicine for clinical diagnosis and monitoring. Because of increasing advances in electronic and mobile technologies, there is an increasing demand for chronic implantable pressure-sensing systems. The biologic environment presents a host of engineering issues that should be considered when developing implantable pressure sensors and implantable electronics including reducing implant size to match anatomic size constraints.

### SUMMARY

Fully implantable sensors for chronic monitoring of pressures represent a potential holy grail for chronic disease management by providing continuous, accurate relative pressure measurements that can be wirelessly transmitted to enable real-time remote diagnostics and therapeutics. Combined with the use of mobile technologies, the inventive implanted sensor systems disclosed herein have the potential to transform management of some of the world's most prevalent and impactful chronic diseases.

An inventive implantable pressure-sensing device may include a pressure-sensing element with a transmitter to transmit the pressure data acquired with the pressure-sensing element to an external element. The pressure data may include, but is not limited to, cardiovascular pressure data, intraocular pressure data, and/or bladder pressure data. The implantable pressure-sensing device may be configured to be implanted in a mammalian subject. The design of the implantable pressure-sensing device may account for interactions between the implanted pressure sensor system and the body, including issues of size, biocompatibility, invasiveness, patient comfort, and ease of use.

Underlying the design of implanted pressure sensor systems is the desire to reduce or minimize the size of the implanted pressure system. A smaller implanted pressure system can be placed in anatomic locations that were previously unimaginable. Similarly, making the implant smaller improves patient comfort, tolerance, and ease of use. It also reduces risks of complications, such as device infection, and expands the clinical applications that can be addressed by the implanted systems.

The inventive implantable pressure sensor systems disclosed herein may have pressure-sensing elements that are physically separated from the electronic components to increase the degree of device miniaturization. For instance, an inventive implantable pressure sensor can include a capacitive or piezoresistive sensing element that is connected to electronics for operating the sensor via a thin, flexible interconnecting bridge. These electronics may include, without limitation, a microcontroller, voltage amplifiers resistors, capacitors, memory units, RFID modules, batteries, and so on. The pressure-sensing element can have a sensing area of about 0.5 mm by 0.5 mm to about 2 mm by 2 mm, and the electronics can be nearly as small (e.g., about 1 mm by 1 mm to about 5 mm by 5 mm). Separating the small pressure-sensing element from the electronics reduces the impact of the implantable pressure sensor system's overall size by allowing the smallest component, the pressure-sensing element, to be separated from the larger electronics assembly without interfering with the implantable pressure sensor system's functions. The pressure-sensing element and electronics assembly can be placed into two separate defined anatomic spaces, lessening the risks of complication related to implant size. The implantable pressure sensor system's interfaces can be coated with a multilayer hermetic coating, which is applied in such a way that pressure transduction and wireless transmission of pressure data is not compromised.

An inventive implantable pressure-sensing device can include a pressure-sensing element, readout electronics, and a bridge connecting and operably coupling the pressure-sensing element to the readout electronics. The pressure-sensing element is configured to be implanted in and to measure pressure within a first anatomic space in a human body. The readout electronics are configured to be implanted in a second anatomic space in the human body and to receive data from the pressure-sensing element.

The implantable pressure-sensing device may also include a conformal coating disposed on the pressure-sensing element to hermetically seal the pressure-sensing element. This conformal coating including alternating layers of ceramic (e.g., SiOₓ) and polymer (e.g., parylene C). The pressure-sensing element can include a rigid substrate, a pressure-sensing membrane supported by the rigid substrate, and a filling material disposed on the pressure-sensing membrane and encapsulated by the conformal coating.

In some examples, the pressure-sensing element and readout electronics may be configured to be implanted inside of the eye and on top of the eye, respectively, in which case the pressure-sensing element is configured to measure intraocular pressure. An example implantable pressure-sensing device can also include an intraocular drainage device, in which case the pressure-sensing element can be positioned to measure pressure of bodily fluid running through the intraocular drainage device and the readout electronics are positioned on a surface of the intraocular drainage device.

In other example implantable pressure-sensing devices, the pressure-sensing element and readout electronics may be configured to be implanted in the left and right atria, respectively, of a heart, in which case the pressure-sensing element is configured to measure pressure in the left atrium. Such an implantable pressure-sensing device can also include an oxygen sensor configured to be implanted in the left atrium. In some cases, the pressure-sensing element is a first pressure-sensing element, and the implantable pressure-sensing device also includes a second pressure-sensing element configured to be implanted in the right atrium and to measure pressure in the right atrium. In these cases, the readout electronics can determine a differential pressure between the left and right atria based on measurements by the first and second pressure-sensing elements.

In still other inventive implantable pressure-sensing devices, the pressure-sensing element and readout electronics may be configured to be implanted inside and outside, respectively, of the bladder. In these cases, the pressure-sensing element is configured to measure pressure in the bladder.

For all of the configurations disclosed herein, the pressure-sensing element and readout electronics may be placed endoscopically under direct vision.

In some aspects, the techniques described herein relate to an implantable pressure-sensing device including a pressure-sensing element configured to be implanted in and to measure pressure within a first anatomic space in a human body, readout electronics configured to be implanted in a second anatomic space in the human body and to receive data from the pressure-sensing element, and a bridge connecting and operably coupling the pressure-sensing element to the readout electronics.

In some aspects, the techniques described herein relate to an implantable pressure-sensing device further including a conformal coating disposed on the pressure-sensing element to hermetically seal the pressure-sensing element, the conformal coating including alternating layers of ceramic and polymer.

In some aspects, the techniques described herein relate to an implantable pressure-sensing device wherein the ceramic includes SiOx and the polymer includes parylene.

In some aspects, the techniques described herein relate to an implantable pressure-sensing device wherein the pressure-sensing element includes a rigid substrate, a pressure-sensing membrane supported by the rigid substrate, and a filling material disposed on the pressure-sensing membrane and encapsulated by the conformal coating.

In some aspects, the techniques described herein relate to an implantable pressure-sensing device wherein the filling material is a biocompatible gel.

In some aspects, the techniques described herein relate to an implantable pressure-sensing device wherein the pressure-sensing membrane includes a plurality of layers.

In some aspects, the techniques described herein relate to an implantable pressure-sensing device wherein the plurality of layers includes a reference layer and a sensing layer.

In some aspects, the techniques described herein relate to an implantable pressure-sensing device wherein the bridge includes at least one wire.

In some aspects, the techniques described herein relate to an implantable pressure-sensing device wherein the bridge further includes a biocompatible polymer to support the at least one wire.

In some aspects, the techniques described herein relate to an implantable pressure-sensing device wherein the bridge is 1 mm to 100 mm long.

In some aspects, the techniques described herein relate to an implantable pressure-sensing device wherein the readout electronics include a controller, a power source, and a wireless transceiver.

In some aspects, the techniques described herein relate to an implantable pressure-sensing device wherein the readout electronics further includes an antenna.

In some aspects, the techniques described herein relate to an implantable pressure-sensing device wherein the power source includes a rechargeable battery.

In some aspects, the techniques described herein relate to an implantable pressure-sensing device wherein the first anatomic space is inside of an eye, the second anatomic space is on top of the eye, and the pressure-sensing element is configured to measure intraocular pressure.

In some aspects, the techniques described herein relate to an implantable pressure-sensing device, further including an intraocular drainage device, wherein the pressure-sensing element is positioned to measure pressure of bodily fluid running through the intraocular drainage device and the readout electronics are positioned on a surface of the intraocular drainage device.

In some aspects, the techniques described herein relate to an implantable pressure-sensing device wherein the first anatomic space is a left atrium of a heart, the second anatomic space is a right atrium of the heart, and the pressure-sensing element is configured to measure pressure in the left atrium.

In some aspects, the techniques described herein relate to an implantable pressure-sensing device further including an oxygen sensor configured to be implanted in the left atrium.

In some aspects, the techniques described herein relate to an implantable pressure-sensing device wherein the pressure-sensing element is a first pressure-sensing element, and further including a second pressure-sensing element configured to be implanted in the right atrium and to measure pressure in the right atrium, wherein the readout electronics are configured to determine a differential pressure between the left atrium and the right atrium based on measurements by the first pressure-sensing element and the second pressure-sensing element.

In some aspects, the techniques described herein relate to an implantable pressure-sensing device wherein the implantable pressure-sensing device is integrated into an Amplatzer device or an inter-atrial shunting device.

In some aspects, the techniques described herein relate to an implantable pressure-sensing device wherein the first anatomic space is a first location in a right atrium of a heart, the second anatomic space is a second location in the right atrium of the heart, and the pressure-sensing element is configured to measure pressure in the right atrium.

In some aspects, the techniques described herein relate to an implantable pressure-sensing device wherein the implantable pressure-sensing device is integrated into an Amplatzer device or an inter-atrial shunting device.

In some aspects, the techniques described herein relate to an implantable pressure-sensing device wherein the first anatomic space is a first location on a wall of a pulmonary artery, the second anatomic space is a second location on the wall of the pulmonary artery, and the pressure-sensing element is configured to measure pressure in the pulmonary artery.

In some aspects, the techniques described herein relate to an implantable pressure-sensing device wherein the implantable pressure-sensing device is integrated into a stent.

In some aspects, the techniques described herein relate to an implantable pressure-sensing device wherein the first anatomic space is an aneurysm sac, the second anatomic space is an artery, and the pressure-sensing element is configured to measure pressure in the aneurysm sac.

In some aspects, the techniques described herein relate to an implantable pressure-sensing device wherein the implantable pressure-sensing device is integrated into a stent.

In some aspects, the techniques described herein relate to an implantable pressure-sensing device wherein the first anatomic space is an outer wall of a blood vessel, the second anatomic space is a tissue below a cutis, and the pressure-sensing element is configured to measure pressure in the blood vessel.

In some aspects, the techniques described herein relate to an implantable pressure-sensing device wherein the first anatomic space is an inside of a bladder, the second anatomic space is outside of the bladder, and the pressure-sensing element is configured to measure pressure in the bladder.

In some aspects, the techniques described herein relate to an implantable pressure-sensing device wherein the implantable pressure-sensing device is integrated into an Amplatzer device or a shunt.

In some aspects, the techniques described herein relate to an implantable pressure-sensing device wherein the first anatomic space is an inside a graft, the second anatomic space is outside the graft, and the pressure-sensing element is configured to measure pressure in the graft.

In some aspects, the techniques described herein relate to a method of measuring pressure in a mammalian subject with an implanted cardiovascular pressure sensing system, the method including collecting pressure data inside of a first anatomic space in a human body with a pressure-sensing element, transferring the pressure data via a bridge to readout electronics implanted in a second anatomic space in the human body, and wirelessly transferring the pressure data from the readout electronics to a device external to the mammalian subject.

In some aspects, the techniques described herein relate to a method further including wirelessly recharging a power source of the implanted cardiovascular pressure sensing system.

In some aspects, the techniques described herein relate to a method wherein the first anatomic space is inside of an eye, the second anatomic space is on top of the eye, and the pressure data is intraocular pressure data.

In some aspects, the techniques described herein relate to a method wherein the first anatomic space is a left atrium of a heart, the second anatomic space is a right atrium of the heart, and the pressure data is cardiovascular pressure data.

In some aspects, the techniques described herein relate to a method further including collecting oxygen measurements inside of the left atrium of the heart with the implanted cardiovascular pressure sensing system.

In some aspects, the techniques described herein relate to a method wherein the pressure-sensing element is a first pressure-sensing element, and further including: collecting pressure data inside of the right atrium of the heart with a second pressure-sensing element, and determining a differential pressure between the first pressure-sensing element and the second pressure-sensing element.

In some aspects, the techniques described herein relate to a method wherein the first anatomic space is an inside of a bladder, the second anatomic space is outside of the bladder, and the pressure data is bladder pressure data.

In some aspects, the techniques described herein relate to a method of implanting a pressure-sensing device in a mammalian subject, the method including connecting a pressure-sensing element and readout electronics with a bridge, implanting the pressure-sensing element in a first anatomic space in a human body, and implanting readout electronics in a second anatomic space in the human body.

In some aspects, the techniques described herein relate to a method wherein the bridge is 1 mm to 100 mm long.

In some aspects, the techniques described herein relate to a method wherein the first anatomic space is inside of an eye, the second anatomic space is on top of the eye, and the pressure-sensing element is configured to measure intraocular pressure.

In some aspects, the techniques described herein relate to a method wherein the first anatomic space is a left atrium of a heart, the second anatomic space is a right atrium of the heart, and the pressure-sensing element is configured to measure pressure in the left atrium.

In some aspects, the techniques described herein relate to a method wherein the pressure-sensing element and readout electronics are integrated into an Amplatzer device or an inter-atrial shunting device and the implanting the pressure-sensing element and readout electronics further includes implanting the Amplatzer device or the inter-atrial shunting device.

In some aspects, the techniques described herein relate to a method wherein the first anatomic space is a first location in a right atrium of a heart, the second anatomic space is a second location in the right atrium of the heart, and the pressure-sensing element is configured to measure pressure in the right atrium.

In some aspects, the techniques described herein relate to a method wherein the pressure-sensing element and readout electronics are integrated into an Amplatzer device or an inter-atrial shunting device and the implanting the pressure-sensing element and readout electronics further includes implanting the Amplatzer device or the inter-atrial shunting device.

In some aspects, the techniques described herein relate to a method wherein the first anatomic space is a first location on a wall of a pulmonary artery, the second anatomic space is a second location on the wall of the pulmonary artery, and the pressure-sensing element is configured to measure pressure in the pulmonary artery.

In some aspects, the techniques described herein relate to a method wherein the pressure-sensing element and readout electronics are integrated into a stent and the implanting the pressure-sensing element and readout electronics further includes implanting the stent.

In some aspects, the techniques described herein relate to a method wherein the first anatomic space is an aneurysm sac, the second anatomic space is an artery, and the pressure-sensing element is configured to measure pressure in the aneurysm sac.

In some aspects, the techniques described herein relate to a method wherein the pressure-sensing element and readout electronics are integrated into a stent and the implanting the pressure-sensing element and readout electronics further includes implanting the stent.

In some aspects, the techniques described herein relate to a method wherein the first anatomic space is an outer wall of a blood vessel, the second anatomic space is a tissue below a cutis, and the pressure-sensing element is configured to measure pressure in the blood vessel.

In some aspects, the techniques described herein relate to a method wherein the first anatomic space is an inside of a bladder, the second anatomic space is outside of the bladder, and the pressure-sensing element is configured to measure pressure in the bladder.

In some aspects, the techniques described herein relate to a method wherein the first anatomic space is an inside a graft, the second anatomic space is outside the graft, and the pressure-sensing element is configured to measure pressure in the graft.

In some aspects, the techniques described herein relate to a method wherein the implanting the pressure-sensing element and readout electronics further includes endoscopically implanting the pressure-sensing element and readout electronics under direct vision.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1 depicts an implantable pressure sensor system with a miniaturized pressure-sensing membrane that is connected to distance-separated readout electronics by a thin interconnecting bridge.

FIG. 2 illustrates readout electronics for the implantable pressure sensor system of FIG. 1.

FIG. 3A shows a cross section of the miniaturized pressure-sensing element of FIG. 1 encapsulated in a conformal coating.

FIG. 3B shows a cross section of another embodiment of the miniaturized pressure-sensing element of FIG. 1 encapsulated in a conformal coating.

FIG. 4 is a detailed profile view of a suitable conformal coating for a miniaturized pressure-sensing element.

FIG. 5A illustrates an alternative implantable pressure sensor system.

FIG. 5B illustrates the implantable pressure sensor system of FIG. 5A implanted in a human eye.

FIG. 5C illustrates the implantable pressure sensor system of FIG. 5A implanted in a human eye in another configuration.

FIG. 5D illustrates a close up of the configuration shown in FIG. 5C.

FIG. 5E illustrates another configuration of the implantable pressure sensor system of FIG. 5A implanted in a human eye.

FIG. 6 illustrates an implantable intraocular shunt with an integrated pressure sensor system.

FIG. 7 illustrates an implantable pressure sensor system embedded in a wall of a fluid channel of an implantable intraocular shunt.

FIG. 8 illustrates a thin, flexible bridge connecting electronics to a pressure-sensing element embedded in a wall of a fluid channel.

FIG. 9 illustrates an alternative implantable pressure sensor system for use in an intraocular lens.

FIG. 10 illustrates an implantable pressure sensor system implanted in a human heart, with readout electronics in the right atrial chamber connected to a pressure-sensing element in the left atrial chamber by a thin, flexible bridge.

FIG. 11A illustrates another implantable pressure sensor system integrated with a carrier device and implanted in a human heart.

FIG. 11B illustrates another implantable pressure sensor system with two pressure-sensing elements integrated with a carrier device and implanted in a human heart.

FIG. 11C illustrates another implantable pressure sensor system integrated with a carrier device and implanted in a human heart.

FIG. 12A illustrates an implantable pressure sensor system implanted in a human bladder.

FIG. 12B illustrates an implantable pressure sensor system integrated with a carrier device and implanted in a human bladder.

FIG. 12C illustrates another configuration of an implantable pressure sensor system implanted in a human bladder.

FIG. 13 illustrates an implantable pressure sensor system implanted in a graft.

FIG. 14A illustrates an implantable pressure sensor system integrated with a carrier device and implanted in an artery.

FIG. 14B illustrates a cross-section of the implantable pressure sensor system shown in FIG. 14A.

FIG. 15 illustrates an implantable pressure sensor system integrated with a carrier device and implanted in an aneurysm sac.

FIG. 16 illustrates a carrier device suitable for use with the implantable pressure sensor system of FIGS. 10 and 11A-11C.

FIG. 17 illustrates an implantable pressure sensor system implanted near a blood vessel.

### DETAILED DESCRIPTION

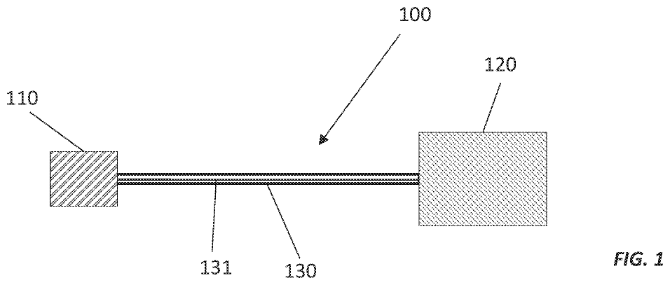

FIG. 1 shows an inventive implantable pressure sensor system 100 suitable for sensing intraocular pressure, cardiovascular pressure, blood pressure, bladder pressure, intra-cranial pressure, and/or orthopedic pressure in a mammalian subject. The implantable pressure sensor system 100 may include a pressure-sensing element 110. The pressure-sensing element may include a pressure-sensing membrane. The pressure-sensing element 110 may be connected to distance-separated readout electronics 120 by a thin, flexible interconnecting bridge 130. The pressure-sensing element 110 may be about 0.5 mm by 0.5 mm by 0.1 mm to about 1 mm by 1 mm by 0.5 mm. The readout electronics 120 may be about 1 mm by 1 mm by 0.4 mm to about 5 mm by 5 mm by 2 mm.

The interconnecting bridge 130 may be about 20 micrometers to about 200 micrometers wide. For example, the interconnecting bridge 130 may about 20 micrometers, about 40 micrometers, about 50 micrometers, about 60 micrometers, about 80 micrometers, about 100 micrometers, about 120 micrometers, about 140 micrometers, about 150 micrometers, about 160 micrometers, about 180 micrometers, or about 200 micrometers wide, including all values in between. The interconnecting bridge 130 may be about 1 mm to about 100 mm long, depending on the anatomic separation between the implanted readout electronics 120 and the implanted pressure-sensing element 110. For example, the interconnecting bridge 130 may be about 1 mm, about 10 mm, about 20 mm, about 30 mm, about 40 mm, about 50 mm, about 60 mm, about 70 mm, about 80 mm, about 90 mm, or about 100 mm long, including all values in between.

The interconnecting bridge 130 may include one or more wires or conductive traces 131 (e.g., 2-4 wires), either alone or supported by a suitable material, such as a silicone or biocompatible polymer (e.g., polyether ether ketone (PEEK)). The optional silicone or biocompatible polymer may provide additional stability and robustness to the one or more wires or conductive traces 131. For example, the optional silicone or biocompatible polymer may wrap around or surround the one or more wires or conductive traces 131. Each wire 131 may be about 20-50 microns in diameter and made of an uncoated biocompatible conductor (e.g., gold) or a conductor, such as copper, coated with a biocompatible coating (e.g., parylene or a multilayer coating, including alternating layers of parylene and a ceramic (e.g., SiOₓ)). The parylene may be parylene C, parylene F, parylene N, parylene D, parylene XY or another suitable type of parylene. The wires or conductive traces 131 connect and carry electrical signals between the pressure-sensing element 110 and the readout electronics 120.

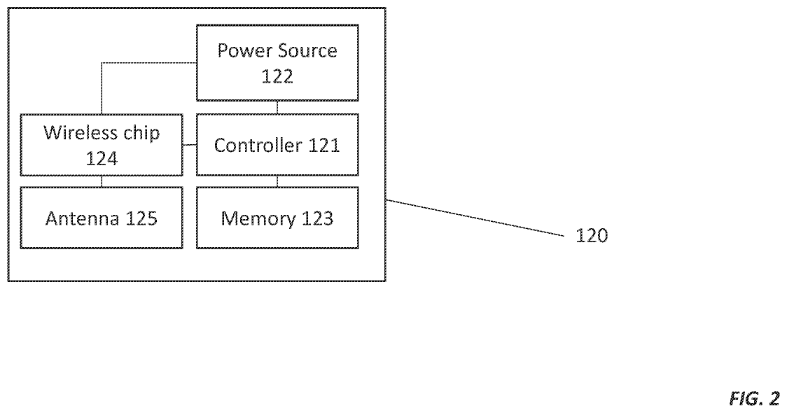

FIG. 2 shows a diagram of the readout electronics 120 suitable for use in the implantable pressure sensor system 100. The readout electronics 120 may include a controller 121, such as a microcontroller or processor, a power source 122 (e.g., a rechargeable battery), memory 123, a wireless transceiver 124 (e.g., a wireless chip), and/or an antenna 125. The readout electronics may also include a sensor (e.g., a second pressure sensor and/or a temperature sensor), resistor, and/or accelerometer (not shown in FIG. 2). The accelerometer may detect or measure body movements and the accelerometer data may be used in processing the pressure data to reduce artifacts associated with body movements. The accelerometer data may be used to differentiate between body movements and pressure waves. Accelerometer data may be used to detect body movement and related pressure artifacts. The accelerometer data may subsequently be used for compensating these artifacts. Due to the relatively short distance of the interconnecting bridge 130, the difference in location between the implanted readout electronics 120 and the implanted pressure-sensing element 110 may be considered negligible for detecting body movement and related pressure artifacts.

The controller 121 may control the implantable pressure sensor system 100 and/or the power source 122. The power source 122 may be a primary battery or a rechargeable battery. Preferably, the management source 122 is a rechargeable battery that is configured to be charged wirelessly so that the implantable pressure sensor system 100 can operate for extended periods while implanted. The readout electronics 120 may also include power management circuitry to control the power source 122. The memory 123 may store data received from the pressure-sensing element 110. The wireless transceiver 124 and/or antenna 125 may wirelessly transmit data from the pressure-sensing element 110 to an external device. For example, the readout electronics 120 may control the operation of the implantable pressure-sensing system 100. For example, the readout electronics 120 may trigger pressure measurements by the pressure-sensing element 110, receive and process pressure measurements by the pressure-sensing element 110, wirelessly transmit raw and/or processed data to external devices (devices outside the patient's body), and/or receive and respond to wireless signals from external devices.

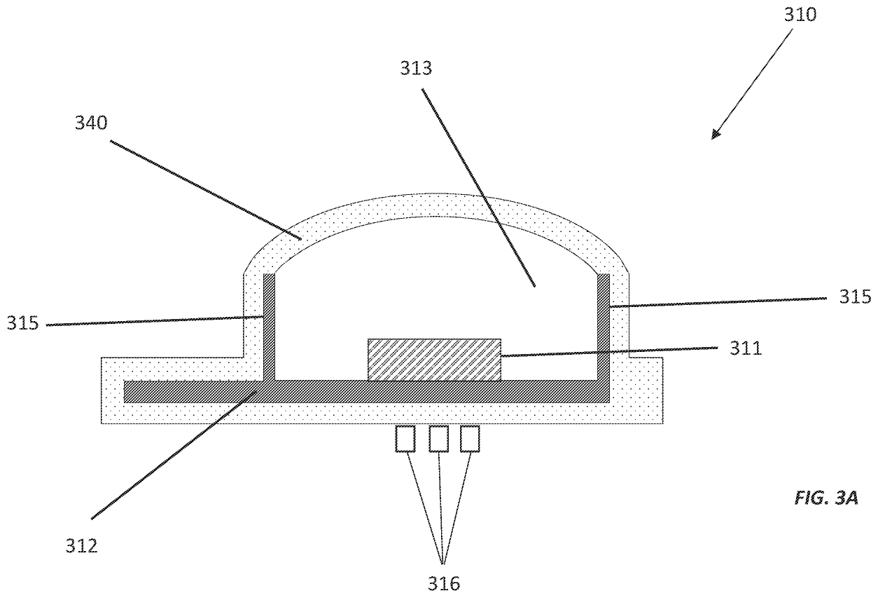

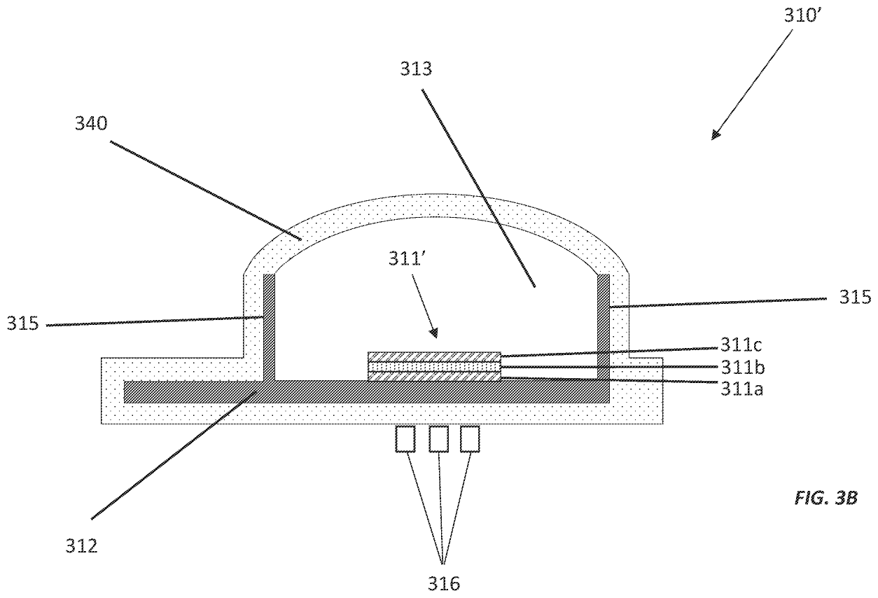

FIGS. 3A and 3B show an example pressure-sensing element 310 suitable for use with the implantable pressure-sensing system 100. The pressure-sensing element 310 may have dimensions of about 0.5 mm×0.5 mm to about 2.0 mm×2.0 mm. For example, the pressure-sensing element 310 may be about 0.6 mm×1.2 mm. As shown in FIG. 3A, the pressure-sensing element 310 may include a pressure-sensing membrane 311 (e.g., a pressure sensor) to measure pressure. The pressure-sensing membrane 311 can be made of silicon or another suitable material and have dimensions of about 0.1 μm×0.1 μm to about 900.0 μm×900.0 μm, for example, about 1 μm×1 μm to about 500 μm×500 μm. The pressure-sensing membrane 311 may have a diameter of about 50 μm to about 500 μm. The pressure-sensing membrane 311 may have a thickness of about 100 nm to about 500 μm. For example, the pressure-sensing membrane 311 may be about 100 nm, about 200 nm, about 300 nm, about 400 nm, about 500 nm, about 600 nm, about 700 nm, about 800 nm, about 900 nm, about 1 μm, about 100 μm, about 200 μm, about 300 μm, about 400 μm, or about 500 μm thick, including all values in between. The pressure-sensing element 310 may be electrically connected to the bridge 130 with bond pads 316. The bond pads 316 may be made of aluminum or gold.

FIG. 3B shows another embodiment of a pressure-sensing element 310′ suitable for use with the implantable pressure-sensing system 100. As described above, the pressure-sensing element 310′ may include a pressure-sensing membrane 311′ (e.g., a pressure sensor) to measure pressure. The pressure-sensing element 310′ may be electrically connected to the bridge 130 with bond pads 316 as described above. The pressure-sensing membrane 311′ can be made of silicon or another suitable material and have dimensions of about 0.1 μm×0.1 μm to about 900 μm×900 μm, for example about 1 μm×1 μm to about 500 μm×500 μm.

The pressure-sensing membrane 311′ may include one or more pressure-sensing membrane layers (e.g., senselets). The pressure-sensing membrane 311′ may include 4 to 100 layers. For example, as shown in FIG. 3B the pressure-sensing membrane 311 may include pressure-sensing membrane layers 311a, 311b, 311c, and so on. Each pressure-sensing membrane layer 311a, 311b, or 311c may have an area in the micrometer range (e.g., about 0.1 μm to about 500 μm). Each pressure-sensing membrane layer 311a, 311b, or 311c may have a thickness of about 100 nm to about 10 μm. For example, each pressure-sensing membrane layer 311a, 311b, or 311c may be about 100 nm, about 200 nm, about 300 nm, about 400 nm, about 500 nm, about 600 nm, about 700 nm, about 800 nm, about 900 nm, about 1 μm, or about 10 μm thick. The pressure-sensing membrane layers 311a, 311b, or 311c may increase linearity and/or sensitivity of the pressure-sensing membrane 311′.

The pressure-sensing membrane layers 311a, 311b, or 311c may include one or more sensing layers and/or one or more reference layers. For example, as shown in FIG. 3B, pressure-sensing membrane layers 311a and 311c are sensing layers and pressure-sensing membrane layers 311b is a reference layer. The one or more reference layer(s) may provide compensation for parasitic effects. The one or more reference layer(s) may be thicker than the one or more sensing layer(s). For example, the one or more reference layer(s) may be about 5 nm thicker, about 10 nm thicker, about 20 nm thicker, about 30 nm thicker, about 40 nm thicker, about 50 nm thicker, about 60 nm thicker, about 70 nm thicker, about 80 nm thicker, about 100 nm thicker, about 200 nm thicker, about 300 nm thicker, about 400 nm thicker, about 500 nm thicker, about 600 nm thicker, about 700 nm thicker, about 800 nm thicker, or about 900 nm thicker than the sensing layer(s).

Pressure-sensing membranes 311 or 311′ may be supported by a rigid carrier substrate 312. The substrate 312 may be made of silicon, ceramic, a polymer (e.g., PEEK), or another suitable substrate material. The substrate 312 may have similar dimensions to the pressure-sensing membrane 311 or 311′. For example, the substrate 312 may have dimensions of about 0.1 μm×0.1 μm to about 900 μm×900 μm, for example about 1 μm×1 μm to about 500 1 μm×500 μm. The rigid carrier substrate 312 may have walls 315 extending around the circumference of the pressure-sensing membrane 311 or 311′. The walls 315 may have a height of about 200 μm to about 1 mm (depending on the height of the pressure-sensing membrane 311 or 311′). The walls 315 may be made of the same material as the substrate 312. Alternatively, the walls 315 may be made of a different material than the substrate 312. For example, the walls 315 may be made of silicon, ceramic, or a polymer (e.g., PEEK).

The pressure-sensing element 310 or 310′ may also covered with a filling material 313. The filling material 313 may be a soft, biocompatible gel (e.g., a silicone gel). Preferably, the filling material 313 is biocompatible and has a Young's modulus (e.g., about 100-1000 kPa) selected so that the filling material 313 can transduce the external pressure to the pressure-sensing membrane 311 or 311′ in a distributed manner. The rigid carrier substrate 312 may ensure a balanced distribution of the external pressure measured by the pressure-sensing membrane 311 or 311′. The pressure-sensing element 310 or 310′ may also be coated with a conformal barrier coating 340. The conformal barrier coating 340 may coat the pressure-sensing membrane 311 or 311′, filling material 313, and rigid carrier substrate 312. The filling material 313 may be pre-treated (e.g., cleaned) in order to ensure good adhesion to the conformal barrier coating 340.

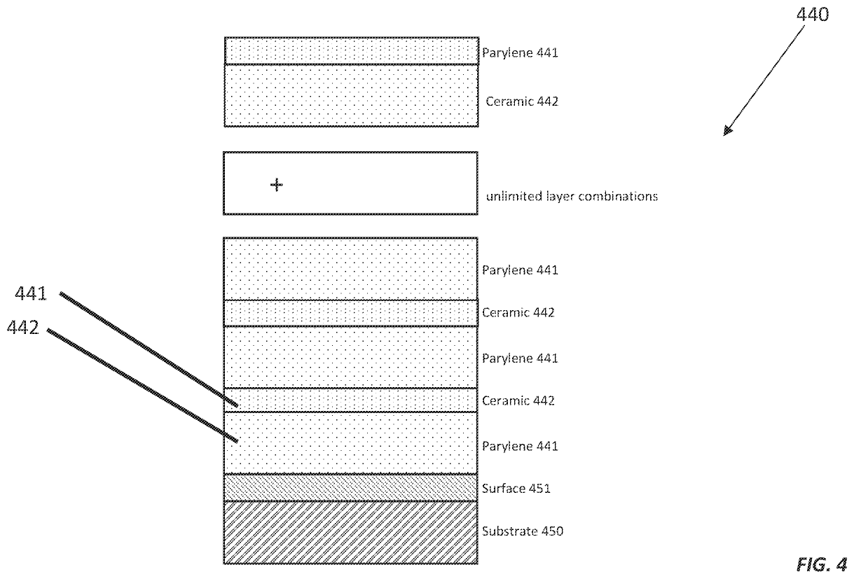

FIG. 4 shows a multilayer conformal barrier coating 440 suitable for coating a surface 451 of a substrate 450. The substrate 450 may be the pressure-sensing element 310 illustrated in FIGS. 3A and 3B, the pressure-sensing element 110 of FIG. 1, the bridge 130, the readout electronics 120, and/or the entire implantable pressure sensor system 100 shown in FIG. 1. This multilayer conformal barrier coating 440 may include alternating polymer layers 441 (e.g., a suitable parylene material, such as parylene C, parylene F, parylene N, parylene D, and/or parylene XY) and ceramic layers 442 (e.g., SiOₓ). The multilayer conformal barrier coating 440 may have a total thickness in the range of about 5 microns to about 100 microns.

In one embodiment, the multilayer conformal barrier coating 440 may extend over the entire implantable pressure sensor system 100, including the bridge 130, readout electronics 120 and the pressure-sensing element 110, 310, or 310′. Alternatively, the bridge 130 and readout electronics 120 can be coated with separate coatings, and/or the bridge can be uncoated and the readout electronics 120 can be coated with a separate coating that is similar or identical to the barrier coating 440. For example, the bridge 130 and readout electronics 120 may be coated parylene, a ceramic (e.g., SiOₓ), and/or the barrier coating 440. One or more layers of the multilayer conformal barrier coating 440 may be treated for surface functionalization (e.g., with hydrophobic or hydrophilic treatment).

Depending on the desired surface properties, the innermost layer of the barrier coating 440 can be either ceramic 442 or polymer 441 with or without dedicated treatment for surface functionalization. Similarly, the outermost layer of the barrier coating 440 can be either ceramic 442 or polymer 441 with or without dedicated treatment for surface functionalization. The number of layers in the barrier coating 440 may depend on the desired application, with respect to hermeticity, thickness and robustness. For example, the barrier coating 440 may include 1-10 layers each of ceramic 442 and polymer 441, with each ceramic layer 442 being on the order of nanometers thick (e.g., about 10-100 nm) and each polymer layer 441 being on the order of microns thick (e.g., about 1-10 μm). The number of layers in the barrier coating 440 may also depend on the portion of the implantable pressure sensor system 100 being coated. For example, the number of layers in the barrier coating 440 covering the pressure-sensing element 110, 310, or 310′ may be different (e.g., thinner) than the number of layers in the barrier coating 440 covering the readout electronics 120 and/or bridge 130. The barrier coating 440 covering the pressure-sensing element 110, 310, or 310′ may include 2-8 layers of each of ceramic 442 and polymer 441.

The combination of filling material 313 and multilayer barrier coating 440 may isolate the pressure-sensing element 310 and/or 110 from the surrounding tissues while allowing the pressure-sensing element 310 and/or 110 to measure pressure accurately and quickly. Optionally, the conformal barrier coating(s) 440 on the pressure-sensing element 310 and/or 110 and readout electronics 120 may also be coated with drug-eluting coating(s) to avoid inflammation. For example, the drug-eluting coating may incorporate drugs and/or other agents that are released at a sustainable rate ranging from a period of 1 week to 6 months. The drug-eluting coating may include inhibitors of fibrosis, including, by example only, TGF-β, other cytokines expressed as mediators of the inflammatory cascade, SMA, and/or integrins.

For more details on implantable pressure sensors and coatings for implantable pressure sensors, see, e.g., U.S. Pat. Nos. 11,497,399 and 11,684,703 to Adams et al and U.S. Pre-Grant Publication No. 2022/0054007 A1 to Adams et al. each of which is incorporated herein by reference in its entirety for all purposes.

### Ophthalmic: Intraocular Pressure Sensing

Intraocular pressure (IOP) sensing is relevant for diagnosis and treatment of eye diseases, e.g., glaucoma. Technical solutions for continuous monitoring of the IOP are not established on the market and should allow for early diagnostics of glaucoma. For more information on glaucoma, see, e.g., U.S. Pat. No. 11,497,399 B2 to Adams et al., which is incorporated herein by reference in its entirety for all purposes.

Existing technical solutions for continuous IOP monitoring rely on indirect measurement of the IOP and are generally attached to contact lenses. For a direct measurement of IOP, the sensing mechanism should be in contact with the anterior or posterior chamber of the eye. Due to the small size of the eyeball and intraocular spaces, and risk of complications, the size of any sensing device for ocular applications should be as small as possible (e.g., about 1-6 mm in diameter).

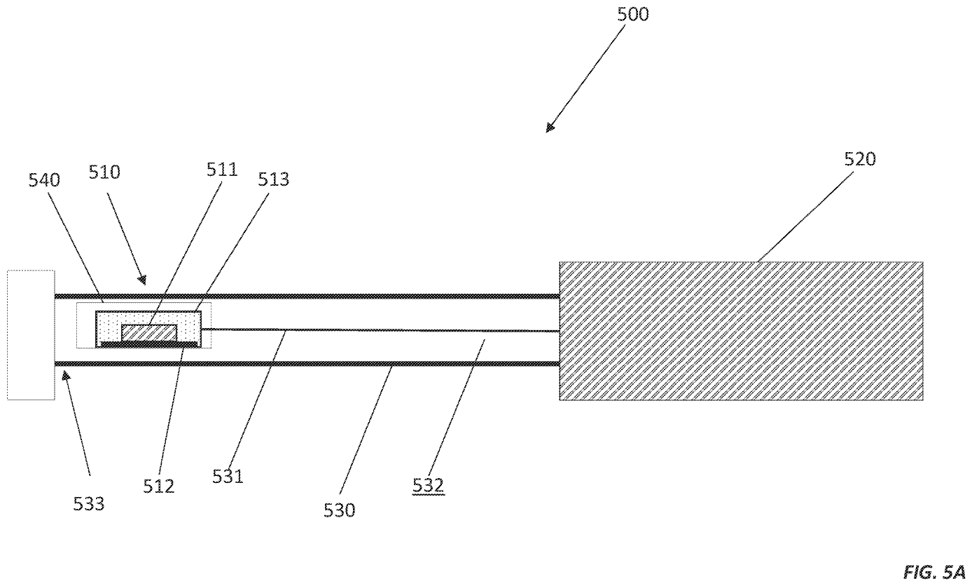

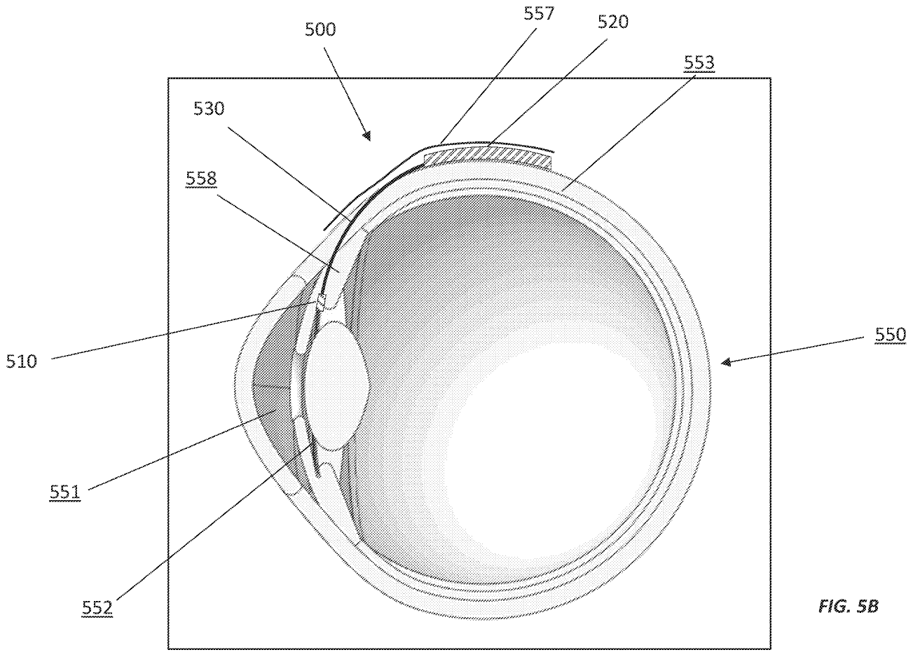

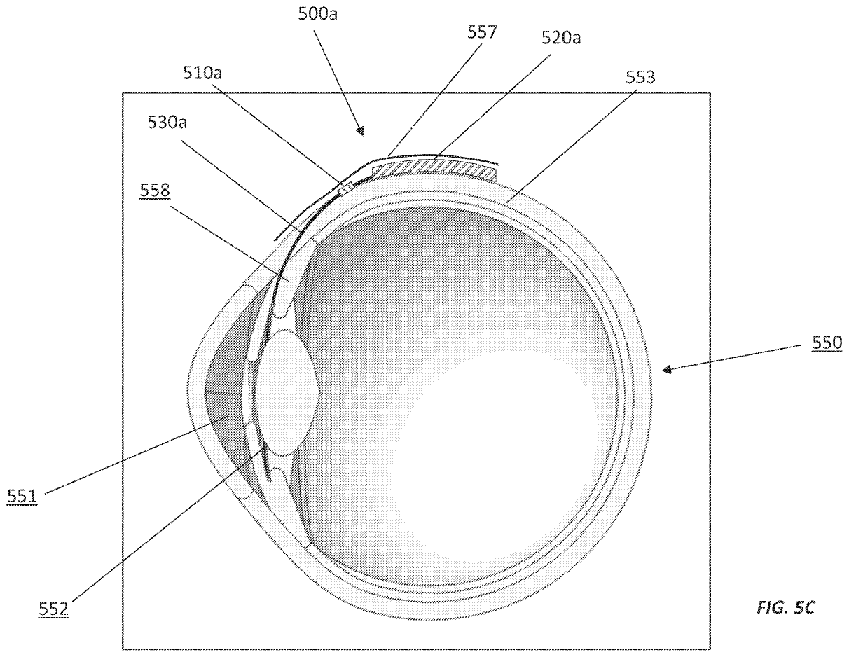

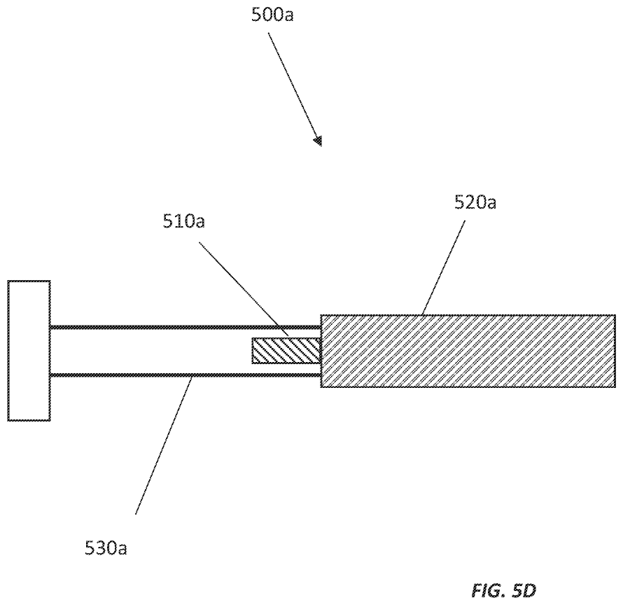

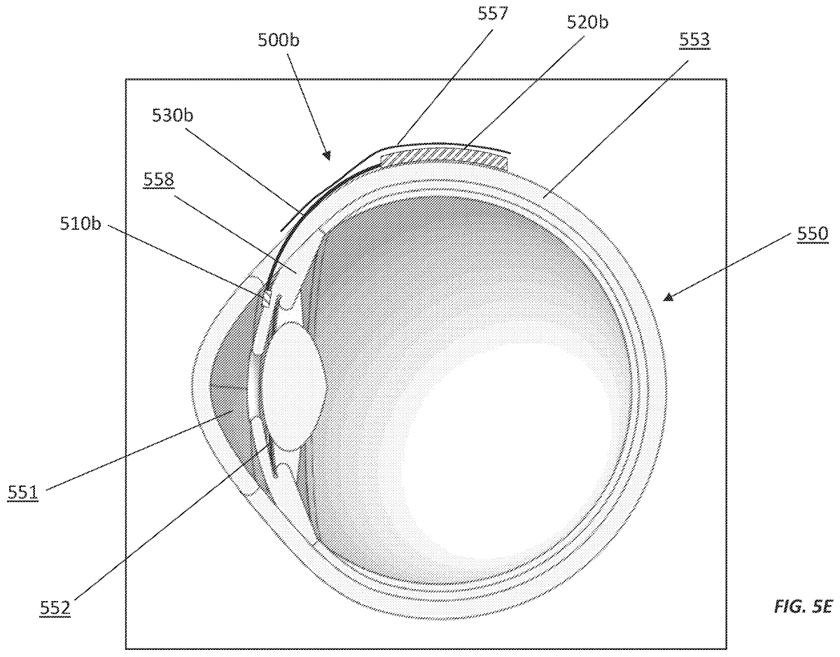

FIGS. 5A and 5B show an inventive implantable pressure sensor system 500 for measuring IOP directly in the anterior chamber 551 of a human eye 550. The implantable pressure sensor system 500 can be implanted in a stand-alone procedure to provide accurate and continuous IOP monitoring. It can also be implanted in conjunction with other procedures, including cataract surgery with implantation of a new intraocular lens and/or minimally invasive glaucoma surgery (MIGS).

The implantable pressure sensor system 500 may include a pressure-sensing element 510 at its distal end physically and communicatively coupled to readout electronics 520 via a bridge 530. The bridge 530 may be made of a substrate 532, such as a silicone elastomer or another suitable biocompatible material as described above. The substrate 532 may be a flexible substrate. For example, the substrate 532 may be a tube of an intra-ocular shunt for drainage. Wires, conductive traces, conductive fibers, or other conductors 531 embedded in or disposed on the substrate 532 may provide electrical connections between the pressure-sensing element 510 and the readout electronics 520. The distal end 533 of the substrate 532 may support the pressure-sensing element 510. The pressure-sensing element 510 may be contained inside of the substrate 532 as shown in FIG. 5A. For example, when the substrate 532 is a tube of a glaucoma shunt for drainage, the pressure-sensing element 510 may be contained inside of the tube of a glaucoma shunt.

The pressure-sensing element 510 may include a pressure-sensing membrane 511 mounted on a rigid substrate 512, a filling material 513, and a multilayer barrier coating 540 as described above with respect to FIGS. 1, 3A, 3B, and 4. The rigid substrate 512 can be mounted to the substrate 532 or molded or fit into an opening or indentation in the substrate 532. For example, in one embodiment, the rigid substrate 512 may be bonded to the substrate 532. The rigid substrate 512 and the substrate 532 may be made of the same material and may be a single part (i.e., a single substrate) such that the substrate 532 acts as the rigid substrate 512 and supports the pressure-sensing membrane 511.

Because the pressure-sensing element 510 is separate from the readout electronics 520, it may be small enough to be implanted in contact with the posterior chamber of the eye 552, as shown in FIG. 5B, or the anterior chamber of the eye 551, as shown in FIG. 5E. As a result, it can measure IOP directly, yielding IOP measurements that are more accurate and more precise than those made with conventional IOP sensors. The readout electronics 520 may be implanted below the conjunctiva 557 in the sub-tenon space or in the supra-choroidal space 553, out of the patient's field of view, with the flexible bridge 530 running between the anterior chamber 551 and choroid 558.

FIGS. 5C and 5D show an example of another configuration of an implantable pressure sensor system 500a. The implantable pressure sensor system 500a may include a pressure-sensing element 510a at its proximal end physically and communicatively coupled to readout electronics 520a via a bridge 530a. The pressure-sensing element 510a may be contained inside of a tube of a glaucoma shunt for drainage as described above. FIG. 5D shows a close up of the configuration shown in FIG. 5C illustrating the pressure-sensing element 510a located at the proximal end adjacent to the readout electronics 520a.

As described above, the bridge 530a may be made of a flexible substrate, such as a silicone elastomer or another suitable biocompatible material as described above. Wires, conductive traces, conductive fibers, or other conductors may be embedded in or disposed on the flexible substrate may provide electrical connections between the pressure-sensing element 510a and the readout electronics 520a. The pressure-sensing element 510a may include a pressure-sensing membrane mounted on a rigid substrate, a filling material, and a multilayer barrier coating as described above with respect to FIGS. 1, 3A, 3B, and 4.

The readout electronics 520a may be implanted below the conjunctiva in the sub-tenon space or in the supra-choroidal space 553, out of the patient's field of view, with the flexible bridge 530 running between the anterior chamber 551 and choroid 558. The readout electronics 520a may include a controller (e.g., a microcontroller or processor), a power source (e.g., a rechargeable battery), memory, a wireless transceiver (e.g., a wireless chip), an antenna, a sensor (e.g., a second pressure sensor and/or a temperature sensor), a resistor, and/or accelerometer as described above with respect to FIG. 2.

FIG. 5E shows an example of another configuration of an implantable pressure sensor system 500b. The implantable pressure sensor system 500b may include a pressure-sensing element 510b at its distal end physically and communicatively coupled to readout electronics 520b via a bridge 530b. The pressure-sensing element 510b may be implanted in the anterior chamber of the eye 551. The pressure-sensing element 510b may be contained inside of a tube of a glaucoma shunt for drainage as described above. The implantable pressure sensor system 500b can measure IOP directly and may yield IOP measurements that are more accurate and more precise than those made with conventional IOP sensors.

As described above, the bridge 530b may be made of a flexible substrate, such as a silicone elastomer or another suitable biocompatible material as described above. Wires, conductive traces, conductive fibers, or other conductors may be embedded in or disposed on the flexible substrate may provide electrical connections between the pressure-sensing element 510b and the readout electronics 520b. The pressure-sensing element 510b may include a pressure-sensing membrane mounted on a rigid substrate, a filling material, and a multilayer barrier coating as described above with respect to FIGS. 1, 3A, 3B, and 4.

The readout electronics 520b may be implanted below the conjunctiva in the sub-tenon space or in the supra-choroidal space 553, out of the patient's field of view, with the flexible bridge 530b running between the anterior chamber 551 and choroid 558. The readout electronics 520b may include a controller (e.g., a microcontroller or processor), a power source (e.g., a rechargeable battery), memory, a wireless transceiver (e.g., a wireless chip), an antenna, a sensor (e.g., a second pressure sensor and/or a temperature sensor), a resistor, and/or accelerometer as described above with respect to FIG. 2.

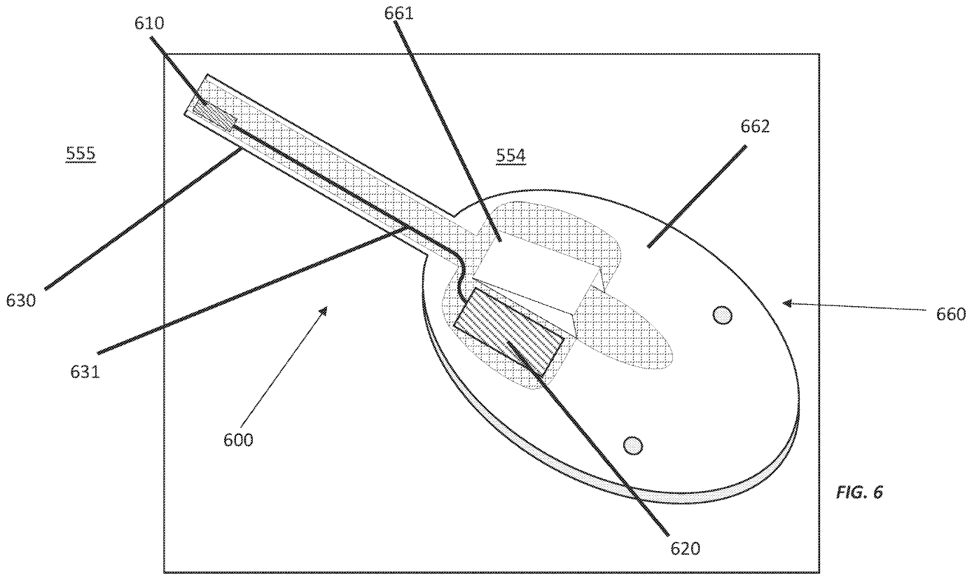

FIG. 6 shows a combination implantable pressure sensor system 600 and intraocular shunt 660 or drainage device (e.g., a glaucoma shunt). The combination implantable pressure sensor system 600 and intraocular shunt 660 may include a pressure-sensing element 610 integrated with or embedded in the intraocular shunt 660, which includes a tube 630 that connects the interior of the eye 554 to the exterior of the eye 555 and is filled with bodily fluid when the shunt 660 is implanted. The shunt 660 may also include a flow restrictor 661 embedded within a flexible holder 662, which is sutured to the eyeball when the shunt 660 is implanted. The flexible holder 662 may be made of polymer and/or silicone.

The pressure-sensing element 610 may be separated from the readout electronics 620 (also as described above) and disposed in or on the tube 630. This arrangement puts the pressure-sensing element 610 in contact with the fluid running through the tube 630 in a nonobstructive way. The pressure-sensing element 610 can be connected to the readout electronics 620 by biocompatible wires or conductive fibers 631 that run lengthwise along the tube 630 and a portion of the shunt 660 or some other attachment (e.g., a bridge or channel connection between the interior 554 and the exterior of the eye 555, such as a fluid path with an entry and an exit). For instance, there can be two or three biocompatible wires 631 made of gold or another suitable conductor with a diameter of about 20-50 micrometers. The pressure-sensing element 610 may include a pressure sensitive membrane as described above, which may be smaller than 1.2 mm×0.6 mm×0.6 mm. For example, the pressure sensitive membrane may have dimensions of about 0.1 μm×0.1 μm to about 900 μm×900 μm, preferably about 1 μm×1 μm to about 500 μm×500 μum. The pressure-sensing element 610 can be located at the distal end, proximal end, or any other position within the tube 630 or outside the tube 630. The pressure-sensing element 610 may include a pressure-sensing membrane mounted on a rigid substrate, a filling material, and a multilayer barrier coating as described above with respect to FIGS. 1, 3A-3B, and 4.

The implantable pressure sensor system 600 and intraocular shunt 660 or drainage device may be implanted in contact with the posterior chamber of the eye 552, as shown in FIGS. 5B and 5C, or the anterior chamber of the eye 551, as shown in FIG. 5E.

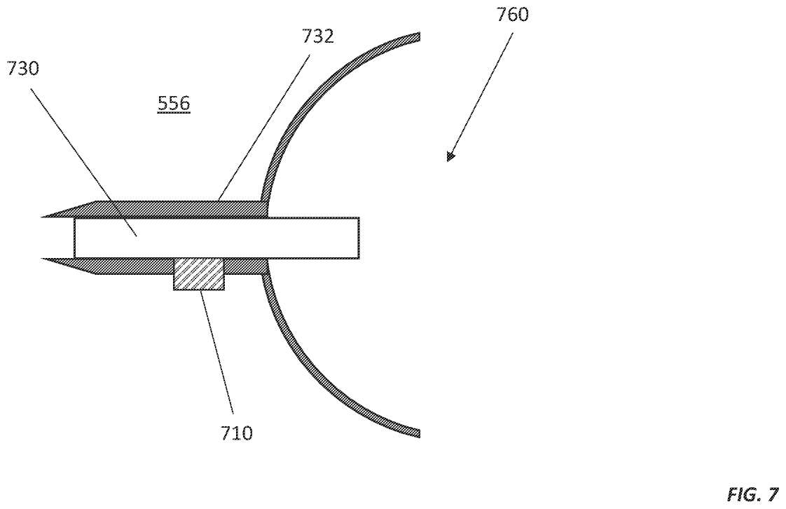

FIG. 7 shows a pressure-sensing element 710 embedded in the proximal portion of an intra-ocular shunt 760 in the anterior chamber 556 of the eye. As shown in FIG. 7, the pressure-sensing element 710 may be embedded in a portion of the channel wall 732 of the fluid channel 730 of the intra-ocular shunt 760. This flexibility makes it possible to adapt the pressure-sensing element 610 and its positioning to other types of products since the pressure in the fluid channel 730 will be the same as long as the fluid channel 730 is exposed to the same fluid. The pressure-sensing element 710 may be connected to readout electronics via biocompatible wires (not shown in FIG. 7) as described above. The pressure-sensing element 710 and intra-ocular shunt 760 may be implanted in contact with the posterior chamber of the eye 552, as shown in FIGS. 5B and 5C, or the anterior chamber of the eye 551, as shown in FIG. 5E. The pressure-sensing element 710 may include a pressure-sensing membrane mounted on a rigid substrate, a filling material, and a multilayer barrier coating as described above with respect to FIGS. 1, 3A, 3B, and 4.

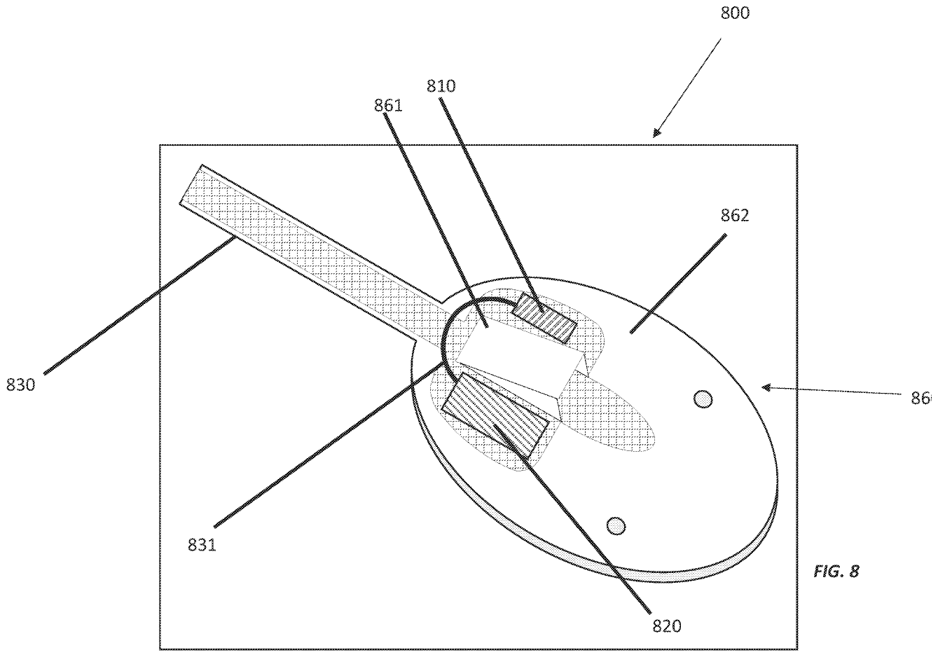

FIG. 8 shows an inventive pressure-sensing device 800 where both the pressure-sensing element 810 and the readout electronics 820 are mounted on the flexible holder or plate 862 of the shunt 860. The shunt 860 may be a glaucoma drainage device. The shunt 860 may also include a flow restrictor 861. The shunt 860 may include a tube 830 that connects the interior of the eye 554 to the exterior of the eye 555 and is filled with bodily fluid when the shunt 860 is implanted. The pressure-sensing element 810 and the readout electronics 820 may separated by a short distance (e.g., about 1 mm to about 10 mm) and connected by a wired bridge or conductive fibers 831. The conductive fibers 831 may be coated as described above. The conductive fibers 831 may also be placed inside of a tube as described above. The pressure-sensing element 810 may be mounted just behind the flow restrictor 861 on the plate 862, which is a common feature of glaucoma devices. After placement, a glaucoma drainage device may be encapsulated by an encapsulating cyst, which may form around the drainage device itself. Since the pressure in the encapsulating cyst is equivalent to the pressure in the anterior chamber 556 of the eye, the pressure-sensing device 800 measures the pressure within the encapsulating cyst by direct contact of the pressure-sensing element 810 with the fluid in the cyst. The pressure-sensing element 810 may include a pressure-sensing membrane mounted on a rigid substrate, a filling material, and a multilayer barrier coating as described above with respect to FIGS. 1, 3A, 3B, and 4.

The inventive pressure-sensing element 810 can be embedded in both valved and non-valved glaucoma drainage devices. For a valved drainage device, the readout electronics 820 may control the valve of the drainage device based on the pressure measured by the pressure-sensing element 810. The pressure-sensing element 810 and intra-ocular shunt 860 may be implanted in contact with the posterior chamber of the eye 552, as shown in FIGS. 5B and 5C, or the anterior chamber of the eye 551, as shown in FIG. 5E.

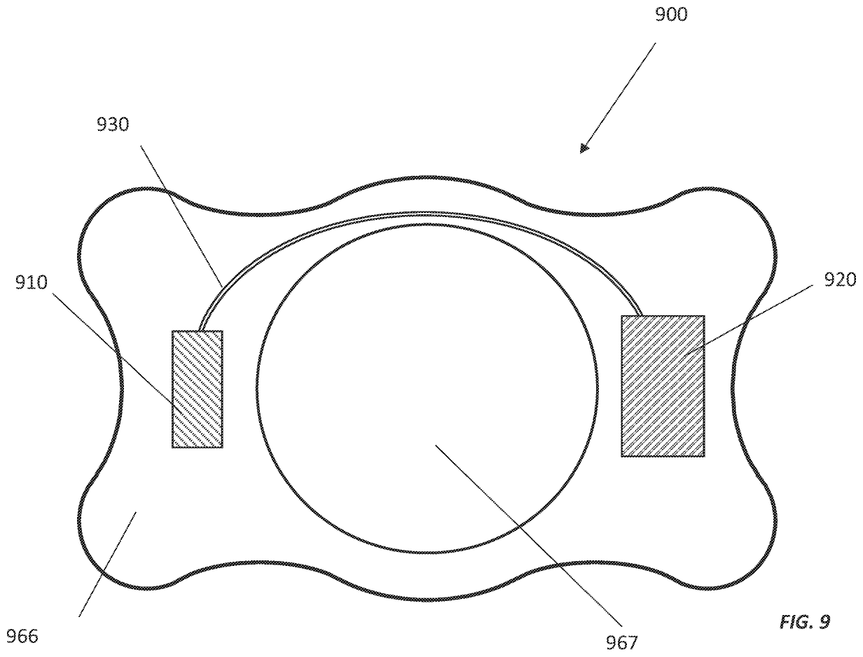

FIG. 9 shows an inventive pressure-sensing device 900 for use with an intraocular lens (IOL) 965. The pressure-sensing device 900 may include a pressure-sensing element 910 and readout electronics 920 as described above. The pressure-sensing element 910 and readout electronics 920 may be mounted on an intraocular lens plate 966. The pressure-sensing element 910 and the readout electronics 920 may be separated by the optical path of the IOL 967 and connected by a bridge 930. The lens plate 966 may act as a carrier to support the pressure-sensing element 910, readout electronics 920, and bridge 930 and for implantation into an eye. The separation of the pressure-sensing element 910 and readout electronics 920 may allow for better use of the space of the lens plate 966 and/or a more balanced weight distribution. The pressure-sensing element 910 may include a pressure-sensing membrane mounted on a rigid substrate, a filling material, and a multilayer barrier coating as described above with respect to FIGS. 1, 3A, 3B, and 4.

As described above, the pressure-sensing elements 510, 610, 710, 810, and 910 and the readout electronics 520, 620, 820, and 920 may be attached to solid substrates, e.g., silicon substrates. The substrate can be glued, over-molded, or clamped between two flexible sheets. The flexible sheets may be made of polymer and/or silicone, for example.

The readout electronics 520, 520a, 520b, 620, 820, and 920 may be in a flat and long hermetically sealed enclosure. The enclosure may have transverse dimensions of between about 1.5 mm×1.5 mm and about 3 mm×3 mm and a thickness or height of about 0.3 mm to about 1.5 mm. The readout electronics 520, 620, 820, and 920 may include one or more integrated circuits for analog-to-digital conversion, signal processing, data transfer/wireless communications with external devices, power management, and control functions. The readout electronics 520, 520a, 520b, 620, 820, and 920 may include a controller (e.g., a microcontroller or processor), a power source (e.g., a rechargeable battery), memory, a wireless transceiver (e.g., a wireless chip), an antenna, a sensor (e.g., a second pressure sensor and/or a temperature sensor), a resistor, and/or accelerometer as described above with respect to FIG. 2.

The pressure-sensing elements 510, 510a, 510b, 610, 710, 810, and 910 and/or the readout electronics 520, 520a, 520b, 620, 820, and 920 may be covered with a thin barrier coating such as the barrier coatings 340 and 440 shown in FIG. 3A/3B and 4, respectively, to ensure hermeticity and sensitivity. As described above, the barrier coating may include at least one layer of parylene and at least one layer of SiOₓ or another suitable combination of polymer (or similar materials, such as acryl materials) and ceramic materials. The barrier coating can be made as described in U.S. Pre-Grant Publication No. 2021/0137379 A1 to Fehr et al., which is incorporated herein by reference in its entirety for all purposes. For example, the barrier coating can be produced by chemical vapor deposition (CVD), sputtering, and/or atomic layer deposition (ALD) and can therefore be produced in a conformal way. The thickness of the barrier coating can range from about 3 micrometers to about 50 micrometers (e.g., about 5 micrometers to about 10 micrometers). The barrier coating may eliminate the need for a hard outer casing, such as a titanium casing, and hence reduce the size, weight, and cost of the implantable pressure sensor system 500, 600, 800, and/or 900.

In one embodiment, the barrier coating may also cover the shunt 660, 760, or 860 and/or the fluid channel 730 or 830. As described above, the barrier coating may include at least one layer of parylene and at least one layer of SiOₓ or another suitable combination of polymer (or similar materials, such as acryl materials) and ceramic materials.

### Heart: Intracardiac Pressure Monitoring

It is of clinical utility to measure pressures in the heart and/or great vessels. For instance, heart failure is a complex clinical syndrome that results from a functional or structural heart disorder impairing ventricular filling or ejection of blood to the systemic circulation. It is a failure to meet the systemic demands of circulation. Heart failure remains a highly prevalent disorder worldwide with a high morbidity and mortality rate. It has an estimated prevalence of 26 million people worldwide and contributes to increased healthcare costs worldwide. The etiology of heart failure varies the treatment plan to some degree; however, most of the treatment recommendations are based on the presence of heart failure alone, regardless of the cause.

Although the pathophysiology and treatment of heart failure is complex, the study of the function and pressures in intracardiac locations such as the right atrium, pulmonary arteries, and/or the left atrium may lead to improved therapy and management of patients with heart failure, particularly those with more advanced stage disease. However, efforts to measure these cited pressures on an outpatient basis are challenging. The pressure-sensing devices disclosed herein may be used to measure cardiovascular pressure, including left atrial pressure, right atrial pressure, and pulmonary artery pressure based on the location of the pressure sensing device in the heart. The pressure-sensing devices disclosed herein may also be used to measure pressure in an aneurysm sac.

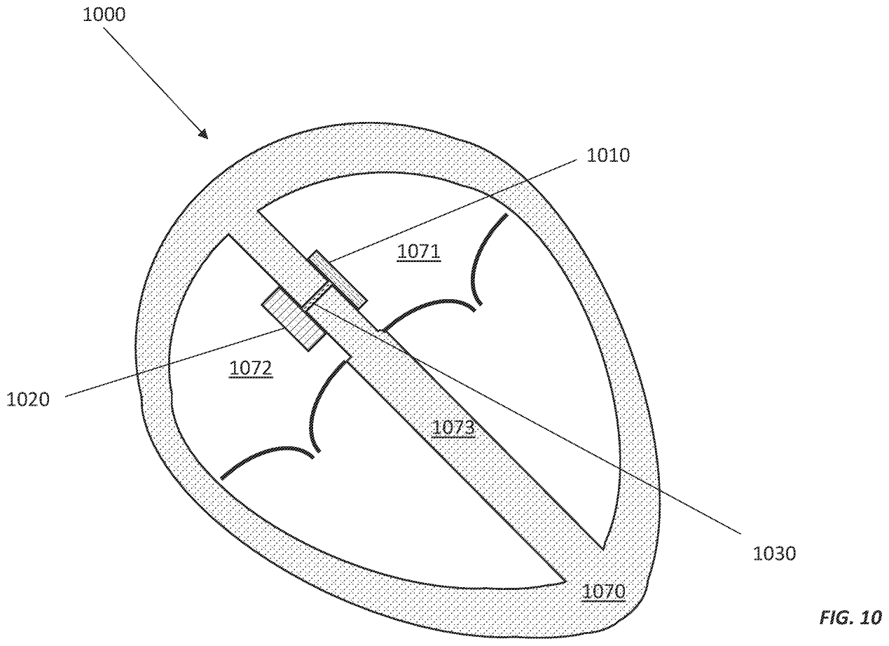

FIG. 10 shows an inventive implantable pressure sensor system 1000 implanted in a human heart 1070 for measuring left atrial pressure. This implantable pressure sensor system 1000 may include a pressure-sensing element 1010 that is separate from the readout electronics 1020 as described above. The pressure-sensing element 1010 and readout electronics 1020 may be in separate anatomic spaces, with the pressure-sensing element 1010 located in the left atrial chamber 1071 and the readout electronics 1020 in the right atrial chamber 1072, in continuity and connection across the atrial septum 1073 of the heart. The pressure-sensing element 1010 and readout electronics 1020 may be connected to each other with a conductive bridge 1030 that extends across the atrial septum 1073. The pressure-sensing element 1010 may include a pressure-sensing membrane mounted on a rigid substrate, a filling material, and a multilayer barrier coating as described above with respect to FIGS. 1, 3A, 3B, and 4.

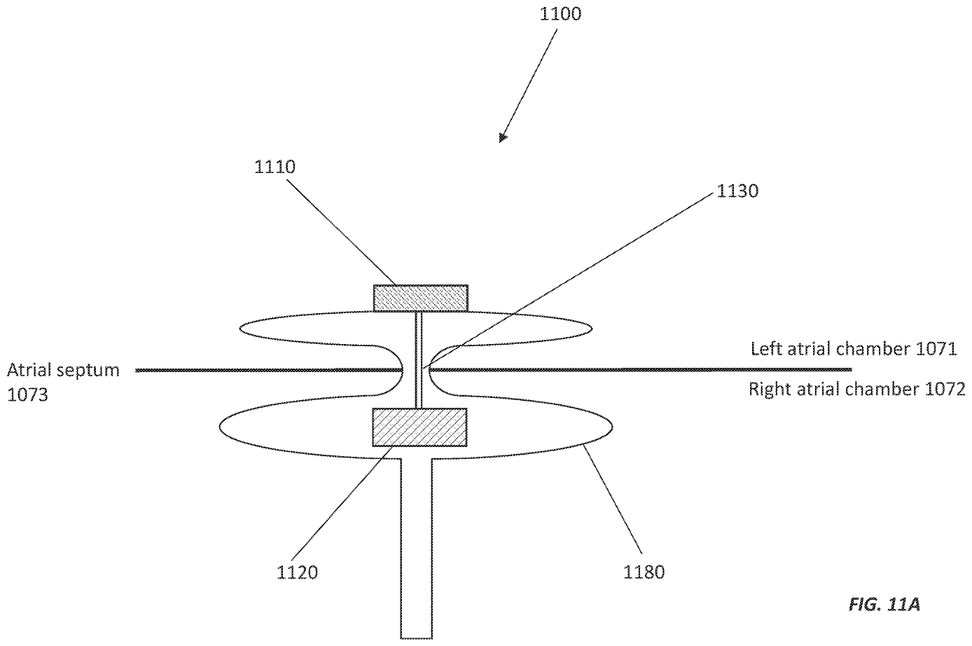

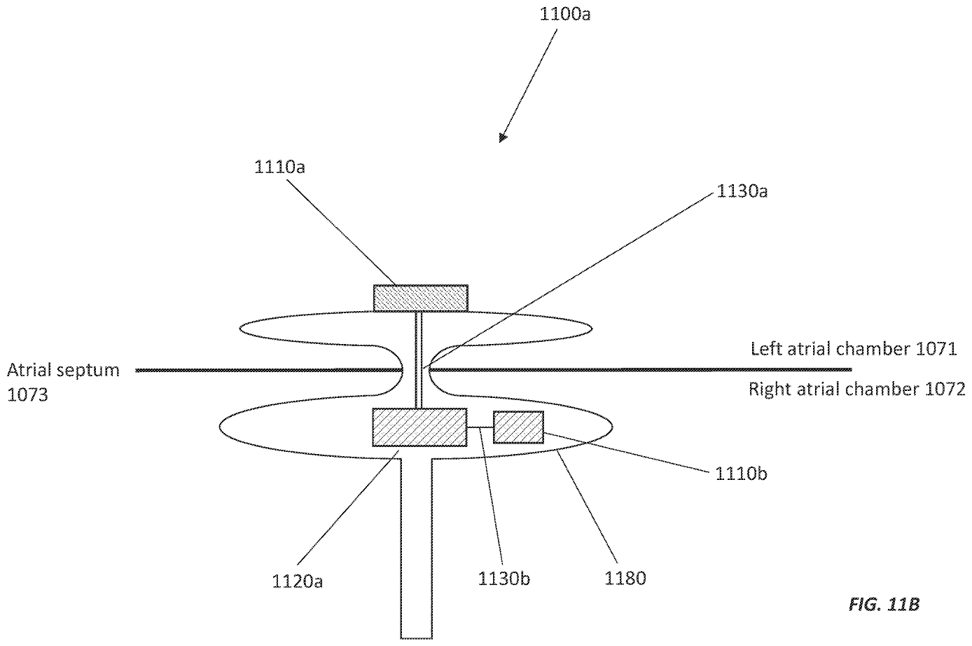

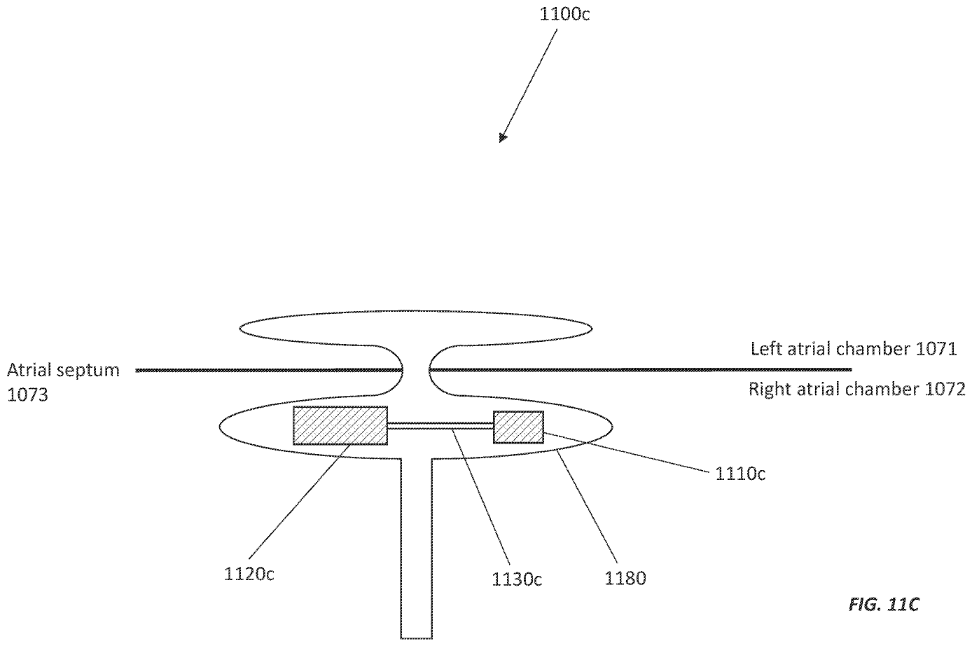

The pressure-sensing element 1010 and the readout electronics 1020 may be supported within a protective carrier device. FIGS. 11A-11C illustrate an implantable pressure-sensing system 1100 integrated with a carrier device 1180. The carrier device 1180 may be an Amplatzer device, also called an Amplatzer septal occluder, which is a percutaneous, transcatheter closure device for atrial septal defects. The Amplatzer device may be a nitinol stent with a polyester covering. Alternatively, the carrier device 1180 may be an inter-atrial shunting device, such as a shunt placed across the inter-atrial septum for shunting blood for patients with congestive heart failure. The pressure-sensing element 1110 may be placed on top of the carrier device 1180 and located in the left atrial chamber 1071 when the carrier device 1180 is implanted in the heart. A bridge 1130 connects the pressure-sensing element 1110 to the readout electronics 1120, which are placed within or on top of the other wing of the carrier device 1180, which is implanted in the right atrial chamber 1072. The pressure-sensing element 1110 may include a pressure-sensing membrane mounted on a rigid substrate, a filling material, and a multilayer barrier coating as described above with respect to FIGS. 1, 3A, 3B, and 4.

The portion of the carrier device 1180 that penetrates through the atrial septum 1073 may be a wire mesh with shape memory characteristics, e.g., a Nitinol mesh, lined with polyester or another suitable material to protect the readout electronics 1120. The implantable pressure-sensing system 1100 may be covered with a thin barrier coating such as barrier coating 340 and/or 440 as shown in FIG. 3A/3B and 4 to ensure hermeticity and sensitivity. Alternatively, individual components of the implantable pressure-sensing system 1100, such as the pressure-sensing element 1110 and the readout electronics 1120, may be covered with a thin barrier coating as described above. If desired, other sensors, such as oxygen sensors, may be placed on the carrier device 1180 next to the pressure-sensing element 1110 in the left atrial chamber 1071. For example, an optical oxygen sensor can be coated with the same coating as the implantable pressure-sensing system 1100, whereas a biochemical oxygen sensor can be coated with a coating with higher oxygen permeability.

Optionally, a second pressure-sensing element may be placed on the carrier device 1180 adjacent to the readout electronics 1120 in the right atrial chamber 1072 for differential pressure measurements (i.e., the difference in the pressures in the left and right atrial pressures). FIG. 11B illustrates an implantable pressure-sensing system 1100a integrated with a carrier device 1180. The implantable pressure-sensing system 1100a may include a first pressure-sensing element 1110a located in the left atrial chamber 1071 and a second pressure-sensing element 1110b located in the right atrial chamber 1071. Both the first and second pressure-sensing elements 1110a and 1110b may be connected to readout electronics 1120a via bridges 1130a and 1130b, respectfully. The pressure-sensing elements 1110a and/or 1110b may include a pressure-sensing membrane mounted on a rigid substrate, a filling material, and a multilayer barrier coating as described above with respect to FIGS. 1, 3A, 3B, and 4.

Optionally the pressure sensing device 1000 or 1100 can be deployed in the right atrial chamber alone, with the pressure sensing device 1000 or 1100 being anchored with a carrier device (e.g., carrier device 1180) with the pressure sensing device 1000 or 1100 located solely on the right side of the atrial septum 1073. FIG. 11C illustrates an implantable pressure-sensing system 1100c integrated with a carrier device 1180 for measuring pressure in the right atrial chamber. The implantable pressure-sensing system 1100c may include a pressure-sensing element 1110c and readout electronics 1120, both of which are located in the right atrial chamber 1072. The pressure-sensing element 1110c may be located in a first location in the right atrial chamber 1072 and the readout electronics 1120 may be located in a second location in the right atrial chamber 1072. The first location in the right atrial chamber 1072 may be any location in the right atrial chamber 1072 and the second location in the right atrial chamber 1072 may be any different location in the right atrial chamber 1072, such that the first location and second location are separated by a distance. The pressure-sensing element 1110c may be connected to readout electronics 1120c via bridge 1130c as described above. The carrier device 1180 (e.g., an Amplatzer device or an inter-atrial shunting device) may be anchored across the septum 1073. The pressure-sensing element 1110c may include a pressure-sensing membrane mounted on a rigid substrate, a filling material, and a multilayer barrier coating as described above with respect to FIGS. 1, 3A-3B, and 4.

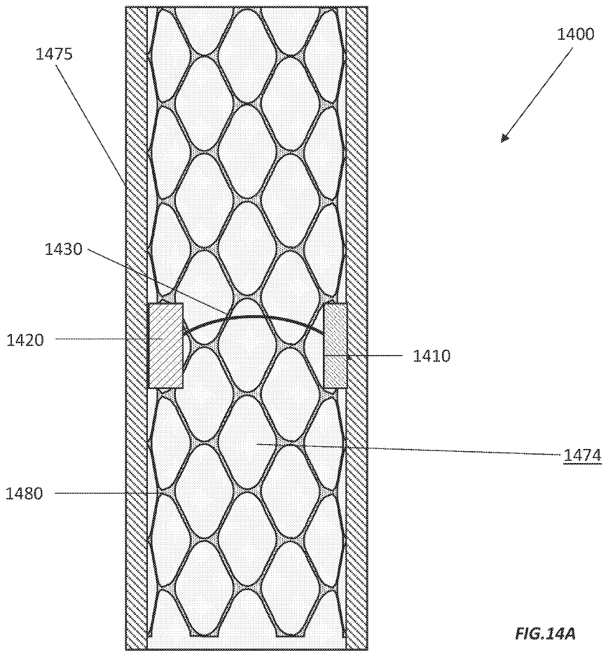

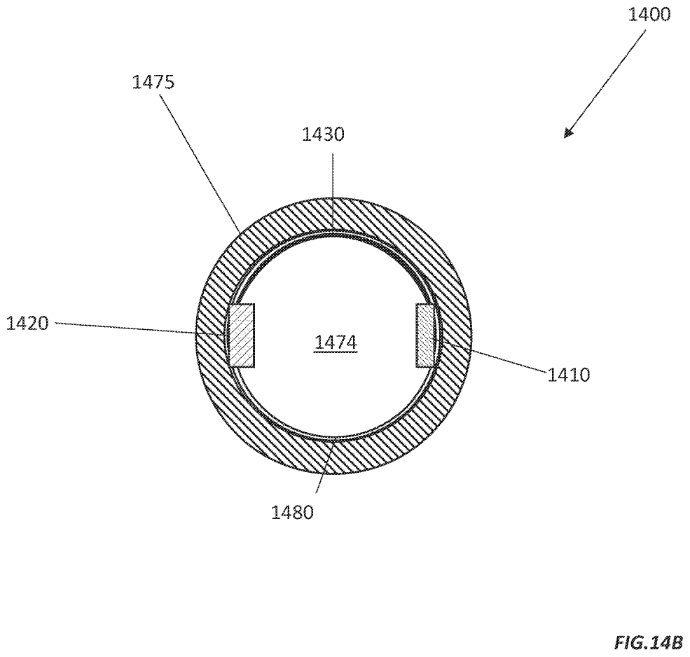

FIGS. 14A-14B illustrate an implantable pressure-sensing system 1400 integrated with a carrier device 1480 for measuring pressure in an artery. The implantable pressure-sensing system 1400 may include a pressure-sensing element 1410 and readout electronics 1420. The pressure-sensing elements 1410 may be connected to readout electronics 1420 via bridge 1430 as described above. The implantable pressure-sensing system 1400 may be integrated with a carrier device 1480 (e.g., a nitinol stent). The carrier device 1480 may be a stent that is positioned in an artery 1474 and configured to hold open the artery 1474 as shown in FIG. 14A. The artery 1474 may be the main, left or right pulmonary artery. The pressure-sensing element 1410 and readout electronics 1420 may be located on or near a wall 1475 of the artery 1474. FIG. 14B shows a cross section view of the artery 1474 containing the pressure-sensing system 1400. The pressure-sensing element 1410 and readout electronics 1420 may be located at different locations on the wall 1475 of the artery 1474 and connected via the bridge 1430. The pressure-sensing element 1410 may be located at a first location on the wall 1475 of the artery 1474 and the readout electronics 1420 may be located at a second location on the wall 1475 of the artery 1474. The first location on the wall 1475 of the artery 1474 may be any location on the wall 1475 of the artery 1474 and the second location on the wall 1475 of the artery 1474 may be any difference location on the wall 1475 of the artery 1474, such that the first location and the second location are separated by a distance. For example, as shown in FIG. 14B, the pressure-sensing element 1410 and readout electronics 1420 may be positioned across from each other along the wall 1475 of the artery 1474 such that the bridge 1430 spans approximately half of the circumference of the artery 1474. The pressure-sensing element 1410 may include a pressure-sensing membrane mounted on a rigid substrate, a filling material, and a multilayer barrier coating as described above with respect to FIGS. 1, 3A-3B, and 4.

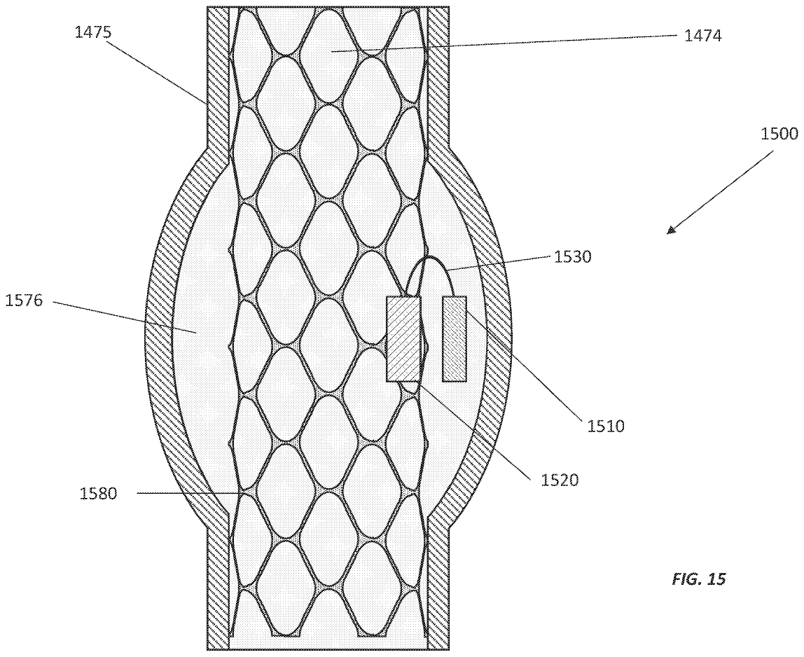

FIG. 15 illustrates an implantable pressure-sensing system 1500 integrated with a carrier device 1580 for measuring pressure in an aneurysm sac. The implantable pressure-sensing system 1500 may include a pressure-sensing element 1510 and readout electronics 1520. The pressure-sensing elements 1510 may be connected to readout electronics 1520 via bridge 1530 as described above. The implantable pressure-sensing system 1500 may be integrated with a carrier device 1580 (e.g., a nitinol stent). The carrier device 1580 may be a stent that is positioned in an artery 1474 and configured to hold open the artery 1474 as shown in FIG. 15. The artery 1474 may be the main, left or right pulmonary artery, for example. As shown in FIG. 15, the pressure-sensing element 1510 may be located in an aneurysm sac 1576 to measure pressure in the aneurysm sac. The readout electronics 1520 may be located on the carrier device 1580 and connected to the pressure-sensing element 1510 via bridge 1530. The pressure-sensing element 1510 may include a pressure-sensing membrane mounted on a rigid substrate, a filling material, and a multilayer barrier coating as described above with respect to FIGS. 1, 3A-3B, and 4.

As described above, bridges 1030, 1130, 1130a, 1130b, 1130c, 1430, and 1530 may be made of a flexible substrate, such as a silicone elastomer or another suitable biocompatible material as described above. Wires, conductive traces, conductive fibers, or other conductors may be embedded in or disposed on the flexible substrate may provide electrical connections between the pressure-sensing elements 1010, 1110, 1110a, 1110b, 1110c, 1410, and 1510 and the readout electronics 1020, 1120, 1120a, 1120c, 1420, and 1520. As described above, the pressure-sensing elements 1010, 1110, 1110a, 1110b, 1110c, 1410, and 1510 and the readout electronics 1020, 1120, 1120a, 1120c, 1420, and 1520 may be attached to solid substrates, e.g., silicon substrates. The substrate(s) can be glued, over-molded, or clamped between flexible sheets.

The readout electronics 1020, 1120, 1120a, 1120c, 1420, and 1520 may be in a flat and long hermetically sealed enclosure. The enclosure may have transverse dimensions of between about 1.5 mm×1.5 mm and about 5 mm×5 mm and a thickness or height of about 0.3 mm to about 2 mm. The readout electronics 1020 and 1120 may include one or more integrated circuits for analog-to-digital conversion, signal processing, data transfer/wireless communications with external devices, power management, and control functions. The readout electronics 1020, 1120, 1120a, 1120c, 1420, and 1520 may include a controller (e.g., a microcontroller or processor), a power source (e.g., a rechargeable battery), memory, a wireless transceiver (e.g., a wireless chip), an antenna, a sensor (e.g., a second pressure sensor and/or a temperature sensor), a resistor, and/or accelerometer as described above with respect to FIG. 2.

The pressure-sensing elements 1010, 1110, 1110a, 1110b, 1110c, 1410, and 1510 and/or the readout electronics 1020, 1120, 1120a, 1120c, 1420, and 1520 may be covered with a thin barrier coating such as barrier coating 340 and/or 440 as shown in FIGS. 3A-3B and 4 to ensure hermeticity and sensitivity. As described above, the barrier coating may include at least one layer of parylene and at least one layer of SiOₓ or another suitable combination of polymer (or similar materials, such as acryl materials) and ceramic materials. The barrier coating can be made as described above. The thickness of the barrier coating can range from about 3 micrometers to about 50 micrometers (e.g., about 5 micrometers to about 10 micrometers). The barrier coating may eliminate the need for a hard outer casing, such as a titanium casing, and hence reduce the size, weight, and cost of the implantable pressure sensor system 1000, 1100, 1100a, 1100c, 1400, and/or 1500. In one embodiment, the barrier coating may be treated for surface functionalization (e.g., with hydrophobic or hydrophilic treatment) as described above. Preferably, the barrier coating for the implantable pressure sensor system 1400 and/or 1500 may have a hydrophilic surface treatment. For example, the pressure-sensing element 1410 and/or 1510, the readout electronics 1420 and/or 1520, and/or the entire implantable pressure sensor system 1400 and/or 1500 may have a hydrophilic surface treatment.

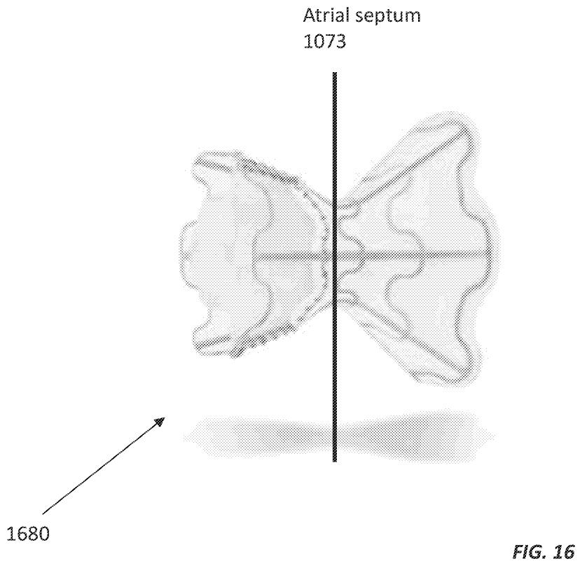

FIG. 16 shows an illustration of an inter-atrial shunting device 1680 that may be used with the pressure sensing devices described above (e.g., pressure sensing device 1000, 1100, 1100a, and/or 1100c). The shunting device 1680 may be placed across the inter-atrial septum 1073 for shunting blood for patients with congestive heart failure.

### Bladder

Globally, over 900 million people suffer from urinary dysfunction from a number of causes, including benign prostatic hyperplasia urinary incontinence and neurogenic bladder. Continuous pressure monitoring based on the natural filling and emptying of the bladder could revolutionize insight into bladder function and improve the treatment of the underlying disease.

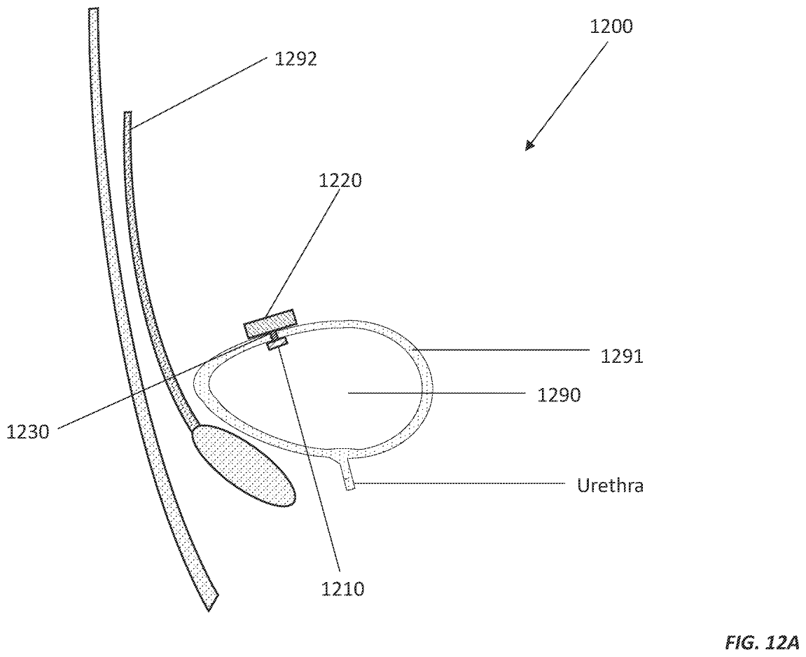

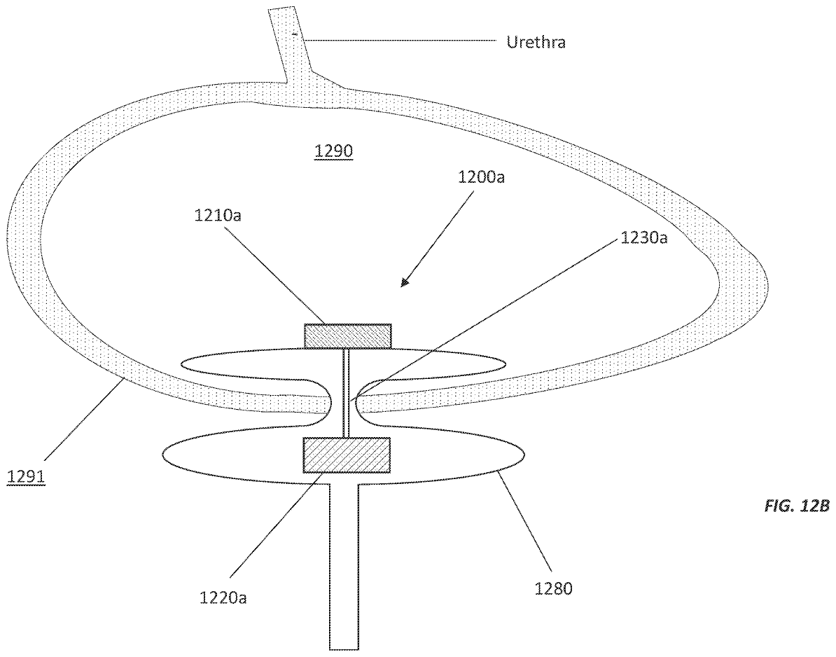

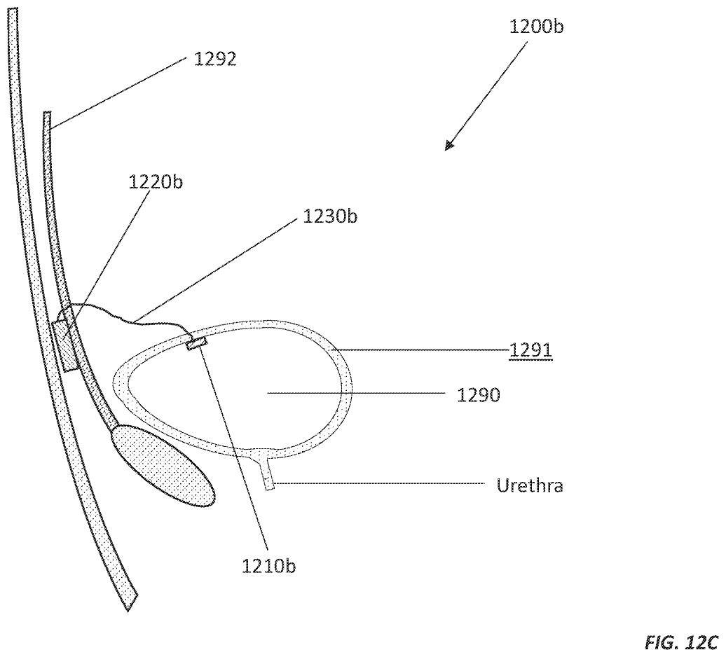

FIG. 12A shows an implantable pressure sensor system 1200 implanted in the bladder 1290 for measuring continuous bladder pressure. The implantable pressure sensor system 1200 may include a pressure-sensing element 1210 and readout electronics 1220 as described above. The pressure-sensing element 1210 and readout electronics 1220 may be packaged separately, connected by a thin bridge 1230, and implanted into separate anatomic spaces. For example, the pressure-sensing element 1210 may be implanted in the bladder itself 1290 while the readout electronics 1220 are implanted outside of the bladder 1290 (e.g., in the peritoneal cavity). The implantable pressure sensor system 1200 may provide continuous bladder pressure data that is wirelessly transmitted to an external device (e.g., via a wireless transmitter and/or antenna in the readout electronics 1220 as described above) for analysis. The implantable pressure sensor system 1200 may increase patient safety and ease of use, while reducing associated risks.

As shown in FIG. 12A, the pressure-sensing element 1210 may be located inside the bladder 1290, whereas the readout electronics 1120 may be located outside the bladder 1290. Different configurations are possible to achieve this separation. For example, in one configuration, shown in FIG. 12A, the pressure-sensing element 1210 may be free floating inside of the bladder 1290, whereas the bridge 1230 can be tunneled submucosally and the readout electronics 1220 can be placed submucousaly in the bladder wall 1291 (e.g., in the submucosal layer of the bladder wall 1291 and/or in the serosa layer of the bladder wall 1291). The pressure-sensing element 1210 may include a pressure-sensing membrane mounted on a rigid substrate, a filling material, and a multilayer barrier coating as described above with respect to FIGS. 1, 3A-3B, and 4.

FIG. 12B shows another example of an implantable pressure sensor system 1200a. The implantable pressure sensor system 1200a may include a pressure-sensing element 1210a, bridge 1230a, and readout electronics 1220, which may be integrated into and protected by a carrier device 1280. The carrier device 1280 may be similar to the carrier device 1180 (e.g., an Amplatzer device or a shunt). The carrier device 1280 can be made from nitinol mesh with shape memory characteristics. When deployed, it may use two discoid-shaped plates that protect the pressure-sensing element 1210a and readout electronics 1220a, while effectively sealing the bladder 1290 where the bladder wall 1291 is perforated to place the readout electronics 1220a on the outside of the bladder wall 1291. In this configuration, the pressure-sensing element 1210a may be located in the bladder 1290 and the read-out electronics 1220a are outside of the bladder wall 1291, and the bladder 1290 is sealed. The pressure-sensing element 1210a is connected to the readout electronics 1220a via a bridge 1230a. The pressure-sensing element 1210a may include a pressure-sensing membrane mounted on a rigid substrate, a filling material, and a multilayer barrier coating as described above with respect to FIGS. 1, 3A-3B, and 4.

FIG. 12C shows another example of an implantable pressure sensor system 1200b. The implantable pressure sensor system 1200b may include a pressure-sensing element 1210b that may be connected to readout electronics 1220b by a bridge 1230b. The bridge 1230b may form a long, sealed catheter, and the readout electronics 1220b may be implanted subcutaneously for easy access (similar to a pacemaker), e.g., in the abdominal wall 1292. The pressure-sensing element 1210b may include a pressure-sensing membrane mounted on a rigid substrate, a filling material, and a multilayer barrier coating as described above with respect to FIGS. 1, 3A-3B, and 4.

In all of these configurations, the sensor and readout electronics can be placed endoscopically under direct vision.

As described above, the bridge 1230, 1230a, or 1230b may be made of a flexible substrate, such as a silicone elastomer or another suitable biocompatible material as described above. Wires, conductive traces, conductive fibers, or other conductors may be embedded in or disposed on the flexible substrate may provide electrical connections between pressure-sensing elements 1210, 1210a, and 1210b and the readout electronics 1220, 1220a, and 1220b. As described above, the pressure-sensing elements 1210, 1210a, and 1210b and the readout electronics 1220, 1220a, and 1220b may be attached to solid substrates, e.g., silicon substrates. The substrate can be glued, over-molded, or clamped between two flexible sheets.

The readout electronics 1220, 1220a, or 1220b may be in a flat and long hermetically sealed enclosure. The enclosure may have transverse dimensions of between about 1.5 mm×1.5 mm and about 5 mm×5 mm and a thickness or height of about 0.3 mm to about 2.0 mm. The readout electronics 1220, 1220a, or 1220b may include one or more integrated circuits for analog-to-digital conversion, signal processing, data transfer/wireless communications with external devices, power management, and control functions. The readout electronics 1220, 1220a, or 1220b may include a controller (e.g., a microcontroller or processor), a power source (e.g., a rechargeable battery), memory, a wireless transceiver (e.g., a wireless chip), an antenna, a sensor (e.g., a second pressure sensor and/or a temperature sensor), a resistor, and/or accelerometer as described above with respect to FIG. 2.

The pressure-sensing element 1210, 1210a, or 1210b and/or the readout electronics 1220, 1220a, or 1220b may be covered with a thin barrier coating such as the barrier coatings 340 and 440 shown in FIGS. 3A-3B and 4, respectively, to ensure hermeticity and sensitivity. As described above, the barrier coating may include at least one layer of parylene and at least one layer of SiOₓ or another suitable combination of polymer (or similar materials, such as acryl materials) and ceramic materials. The barrier coating can be made as described above. The thickness of the barrier coating can range from about 3 micrometers to about 50 micrometers (e.g., about 5 micrometers to about 10 micrometers). The barrier coating may eliminate the need for a hard outer casing, such as a titanium casing, and hence reduce the size, weight, and cost of the implantable pressure sensor system 1200, 1200a, and/or 1200b.

### Graft

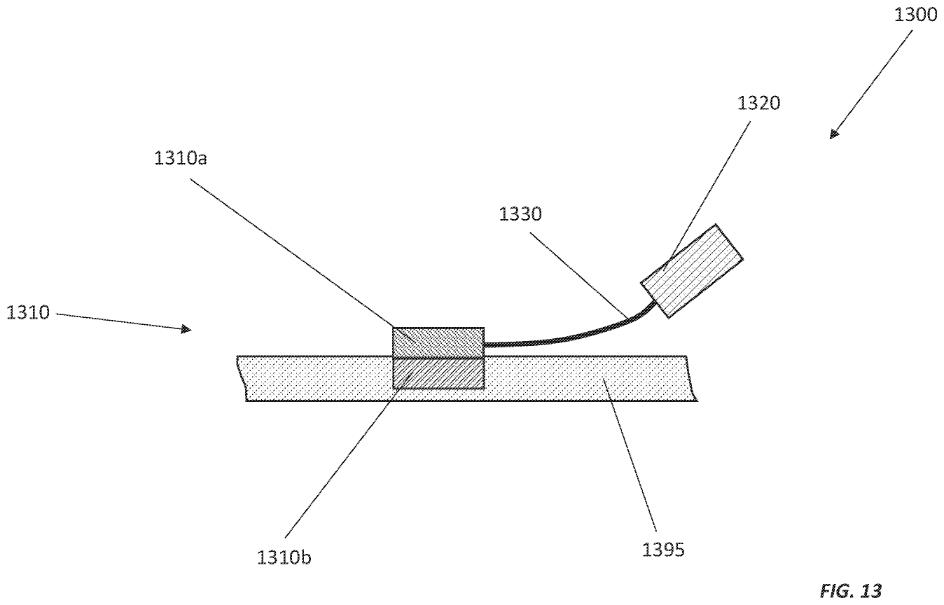

FIG. 13 shows an implantable pressure sensor system 1300 implanted in a graft 1395. The implantable pressure sensor system 1300 may include a pressure-sensing element(s) 1310 and readout electronics 1320 as described above. The pressure-sensing element(s) 1310 and readout electronics 1320 may be connected by a thin bridge 1330. The pressure-sensing element 1310 may include two pressure-sensing elements, pressure-sensing element 1310a and pressure-sensing element 1310b. Pressure-sensing element 1310a may be located on the outside of the graft 1395 and pressure-sensing element 1310b may be located on the inside of the graft 1395. Both pressure-sensing element 1310a and pressure-sensing element 1310b may be connected to the readout electronics 1320 via the bridge 1330. The pressure-sensing element 1310 may measure blood pressure in the graft 1395. Alternatively, pressure-sensing element 1310 may measure differential pressure (e.g., by comparing the pressure between pressure-sensing element 1310a and pressure-sensing element 1310b) to reduce artefacts. The pressure-sensing element 1310, 1310a, and/or 1310b may include a pressure-sensing membrane mounted on a rigid substrate, a filling material, and a multilayer barrier coating as described above with respect to FIGS. 1, 3A, 3B, and 4.

As described above, the bridge 1330 may be made of a flexible substrate, such as a silicone elastomer or another suitable biocompatible material as described above. Wires, conductive traces, conductive fibers, or other conductors may be embedded in or disposed on the flexible substrate may provide electrical connections between the pressure-sensing elements 1310, 1310a, and 1310b and the readout electronics 1320. As described above, the pressure-sensing elements 1310, 1310a, and 1310b and the readout electronics 1320 may be attached to solid substrates, e.g., silicon substrates. The substrate can be glued, over-molded, or clamped between two flexible sheets.

The readout electronics 1320 may be in a flat and long hermetically sealed enclosure. The enclosure may have transverse dimensions of between about 1.5 mmx 1.5 mm and about 5 mm×5 mm and a thickness or height of about 0.3 mm to about 2.0 mm. The readout electronics 1320 may include one or more integrated circuits for analog-to-digital conversion, signal processing, data transfer/wireless communications with external devices, power management, and control functions. The readout electronics 1320 may include a controller (e.g., a microcontroller or processor), a power source (e.g., a rechargeable battery), memory, a wireless transceiver (e.g., a wireless chip), an antenna, a sensor (e.g., a second pressure sensor and/or a temperature sensor), a resistor, and/or accelerometer as described above with respect to FIG. 2.

The pressure-sensing elements 1310, 1310a, and 1310b and/or the readout electronics 1320 may be covered with a thin barrier coating such as the barrier coatings 340 and 440 as shown in FIGS. 3A-3B and 4, respectively, to ensure hermeticity and sensitivity. As described above, the barrier coating may include at least one layer of parylene and at least one layer of SiOₓ or another suitable combination of polymer (or similar materials, such as acryl materials) and ceramic materials. The barrier coating can be made as described above. The thickness of the barrier coating can range from about 3 micrometers to about 50 micrometers (e.g., about 5 micrometers to about 10 micrometers). The barrier coating may eliminate the need for a hard outer casing, such as a titanium casing, and hence reduce the size, weight, and cost of the implantable pressure sensor system 1300.

### Blood Pressure

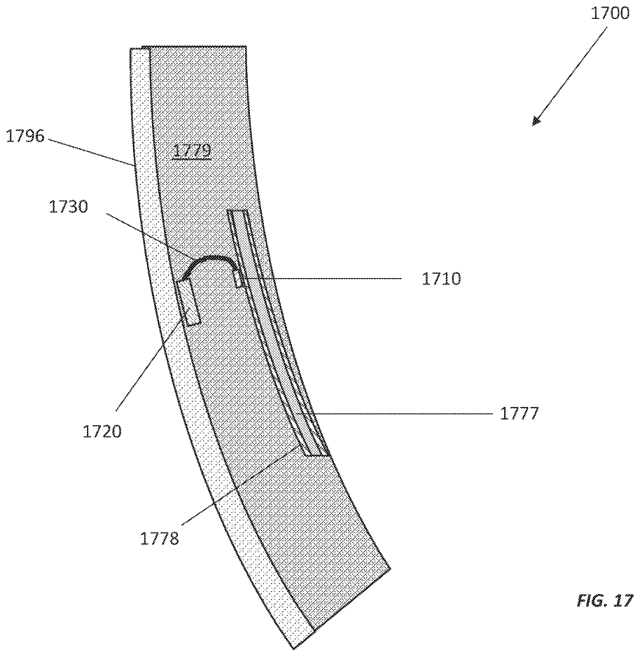

FIG. 17 shows an implantable pressure sensor system 1700 implanted adjacent to a blood vessel 1777 to measure pressure blood pressure. The implantable pressure sensor system 1700 may include a pressure-sensing element 1710 and readout electronics 1720 as described above. The pressure-sensing element 1710 and readout electronics 1720 may be connected by a thin bridge 1730. As shown in FIG. 17, the pressure-sensing element 1710 may be located near or in direct contact with an outer wall 1778 of the blood vessel 1777. For example, the pressure-sensing element 1710 may be secured within 0 mm to 10 mm of the blood vessel 1777. The readout electronics 1720 may be located in the tissue 1779 beneath the cutis 1796 and connected to the pressure-sensing element 1710 via bridge 1730. The pressure-sensing element 1710 may include a pressure-sensing membrane mounted on a rigid substrate, a filling material, and a multilayer barrier coating as described above with respect to FIGS. 1, 3A-3B, and 4.

The blood vessel 1777 may include a natural blood vessel (e.g., an artery, vein, and/or capillary) or a synthetic/artificial blood vessel (e.g., a graft). The artery may include a radial artery, an ulnar artery, a brachial artery, a sub-clavian artery, a renal artery, or the abdominal aorta, for example. The blood vessel may also include a great vessel (e.g., the inferior vena cava, the superior vena cava, a pulmonary artery, a pulmonary vein, or the aorta). The arterial structure may be a natural or synthetic blood vessel in an upper extremity (e.g., an arm), a lower extremity (e.g., a leg), the trunk, and/or the head and neck.

As described above, the bridge 1730 may be made of a flexible substrate, such as a silicone elastomer or another suitable biocompatible material as described above. Wires, conductive traces, conductive fibers, or other conductors may be embedded in or disposed on the flexible substrate may provide electrical connections between the pressure-sensing element 1710, and the readout electronics 1720. As described above, the pressure-sensing element 1710 and the readout electronics 1720 may be attached to solid substrates, e.g., silicon substrates. The substrate can be glued, over-molded, or clamped between two flexible sheets.

The readout electronics 1720 may be in a flat and long hermetically sealed enclosure. The enclosure may have transverse dimensions of between about 1.5 mm×1.5 mm and about 5 mm×5 mm and a thickness or height of about 0.3 mm to about 2.0 mm. The readout electronics 1720 may include one or more integrated circuits for analog-to-digital conversion, signal processing, data transfer/wireless communications with external devices, power management, and control functions. The readout electronics 1720 may include a controller (e.g., a microcontroller or processor), a power source (e.g., a rechargeable battery), memory, a wireless transceiver (e.g., a wireless chip), an antenna, a sensor (e.g., a second pressure sensor and/or a temperature sensor), a resistor, and/or accelerometer as described above with respect to FIG. 2.

The pressure-sensing element 1710 and/or the readout electronics 1720 may be covered with a thin barrier coating such as the barrier coatings 340 and 440 as shown in FIGS. 3A-3B and 4, respectively, to ensure hermeticity and sensitivity. As described above, the barrier coating may include at least one layer of parylene and at least one layer of SiOₓ or another suitable combination of polymer (or similar materials, such as acryl materials) and ceramic materials. The barrier coating can be made as described above. The thickness of the barrier coating can range from about 3 micrometers to about 50 micrometers (e.g., about 5 micrometers to about 10 micrometers). The barrier coating may eliminate the need for a hard outer casing, such as a titanium casing, and hence reduce the size, weight, and cost of the implantable pressure sensor system 1700.

Further examples the use of implantable pressure sensing devices to measure blood pressure can be found in International Patent Application Nos.: PCT/US2023/071396 and PCT/US2023/036409, which are incorporated herein by reference in their entirety for all purposes.

### Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An implantable pressure-sensing device comprising:
a pressure-sensing element configured to be implanted in and to measure pressure within a first anatomic space in a human body;
readout electronics configured to be implanted in a second anatomic space in the human body and to receive data from the pressure-sensing element; and
a bridge connecting and operably coupling the pressure-sensing element to the readout electronics.

2. The implantable pressure-sensing device of claim 1, further comprising:
a conformal coating disposed on the pressure-sensing element to hermetically seal the pressure-sensing element, the conformal coating including alternating layers of ceramic and polymer.

3. The implantable pressure-sensing device of claim 2, wherein the ceramic comprises SiOₓ and the polymer comprises parylene.

4. The implantable pressure-sensing device of claim 2, wherein the pressure-sensing element comprises:
a rigid substrate;
a pressure-sensing membrane supported by the rigid substrate; and
a filling material disposed on the pressure-sensing membrane and encapsulated by the conformal coating.

5. The implantable pressure-sensing device of claim 4, wherein the filling material is a biocompatible gel.

6. The implantable pressure-sensing device of claim 4, wherein the pressure-sensing membrane comprises a plurality of layers.

7. The implantable pressure-sensing device of claim 6, wherein the plurality of layers comprises a reference layer and a sensing layer.

8. The implantable pressure-sensing device of claim 1, wherein the bridge comprises at least one wire.

9. The implantable pressure-sensing device of claim 8, wherein the bridge further comprises a biocompatible polymer to support the at least one wire.

10. The implantable pressure-sensing device of claim 1, wherein the bridge is 1 mm to 100 mm long.

11. The implantable pressure-sensing device of claim 1, wherein the readout electronics comprise a controller, a power source, and a wireless transceiver.

12. The implantable pressure-sensing device of claim 11, wherein the readout electronics further comprises an antenna.

13. The implantable pressure-sensing device of claim 11, wherein the power source comprises a rechargeable battery.

14. The implantable pressure-sensing device of claim 1, wherein the first anatomic space is inside of an eye, the second anatomic space is on top of the eye, and the pressure-sensing element is configured to measure intraocular pressure.

15. The implantable pressure-sensing device of claim 1, further comprising:
an intraocular drainage device,
wherein the pressure-sensing element is positioned to measure pressure of bodily fluid running through the intraocular drainage device and the readout electronics are positioned on a surface of the intraocular drainage device.

16. The implantable pressure-sensing device of claim 1, wherein the first anatomic space is a left atrium of a heart, the second anatomic space is a right atrium of the heart, and the pressure-sensing element is configured to measure pressure in the left atrium.

17. The implantable pressure-sensing device of claim 16, further comprising:
an oxygen sensor configured to be implanted in the left atrium.

18. The implantable pressure-sensing device of claim 16, wherein the pressure-sensing element is a first pressure-sensing element, and further comprising:
a second pressure-sensing element configured to be implanted in the right atrium and to measure pressure in the right atrium,
wherein the readout electronics are configured to determine a differential pressure between the left atrium and the right atrium based on measurements by the first pressure-sensing element and the second pressure-sensing element.

19. The implantable pressure-sensing device of claim 16, wherein the implantable pressure-sensing device is integrated into an Amplatzer device or an inter-atrial shunting device.

20. The implantable pressure-sensing device of claim 1, wherein the first anatomic space is a first location in a right atrium of a heart, the second anatomic space is a second location in the right atrium of the heart, and the pressure-sensing element is configured to measure pressure in the right atrium.

21. The implantable pressure-sensing device of claim 20, wherein the implantable pressure-sensing device is integrated into an Amplatzer device or an inter-atrial shunting device.

22. The implantable pressure-sensing device of claim 1, wherein the first anatomic space is a first location on a wall of a pulmonary artery, the second anatomic space is a second location on the wall of the pulmonary artery, and the pressure-sensing element is configured to measure pressure in the pulmonary artery.

23. The implantable pressure-sensing device of claim 22, wherein the implantable pressure-sensing device is integrated into a stent.

24. The implantable pressure-sensing device of claim 1, wherein the first anatomic space is an aneurysm sac, the second anatomic space is an artery, and the pressure-sensing element is configured to measure pressure in the aneurysm sac.

25. The implantable pressure-sensing device of claim 24, wherein the implantable pressure-sensing device is integrated into a stent.

26. The implantable pressure-sensing device of claim 1, wherein the first anatomic space is an outer wall of a blood vessel, the second anatomic space is a tissue below a cutis, and the pressure-sensing element is configured to measure pressure in the blood vessel.

27. The implantable pressure-sensing device of claim 1, wherein the first anatomic space is an inside of a bladder, the second anatomic space is outside of the bladder, and the pressure-sensing element is configured to measure pressure in the bladder.

28. The implantable pressure-sensing device of claim 27, wherein the implantable pressure-sensing device is integrated into an Amplatzer device or a shunt.

29. The implantable pressure-sensing device of claim 1, wherein the first anatomic space is an inside a graft, the second anatomic space is outside the graft, and the pressure-sensing element is configured to measure pressure in the graft.

30. A method of measuring pressure in a mammalian subject with an implanted cardiovascular pressure sensing system, the method comprising:
collecting pressure data inside of a first anatomic space in a human body with a pressure-sensing element;
transferring the pressure data via a bridge to readout electronics implanted in a second anatomic space in the human body; and
wirelessly transferring the pressure data from the readout electronics to a device external to the mammalian subject.

31. The method of claim 30, further comprising:
wirelessly recharging a power source of the implanted cardiovascular pressure sensing system.

32. The method of claim 30, wherein the first anatomic space is inside of an eye, the second anatomic space is on top of the eye, and the pressure data is intraocular pressure data.

33. The method of claim 30, wherein the first anatomic space is a left atrium of a heart, the second anatomic space is a right atrium of the heart, and the pressure data is cardiovascular pressure data.

34. The method of claim 33, further comprising:
collecting oxygen measurements inside of the left atrium of the heart with the implanted cardiovascular pressure sensing system.

35. The method of claim 33, wherein the pressure-sensing element is a first pressure-sensing element, and further comprising:
collecting pressure data inside of the right atrium of the heart with a second pressure-sensing element; and
determining a differential pressure between the first pressure-sensing element and the second pressure-sensing element.

36. The method of claim 35, wherein the first anatomic space is an inside of a bladder, the second anatomic space is outside of the bladder, and the pressure data is bladder pressure data.

37. A method of implanting a pressure-sensing device in a mammalian subject, the method comprising:
connecting a pressure-sensing element and readout electronics with a bridge;
implanting the pressure-sensing element in a first anatomic space in a human body; and
implanting readout electronics in a second anatomic space in the human body.

38. The method of claim 37, wherein the bridge is 1 mm to 100 mm long.

39. The method of claim 37, wherein the first anatomic space is inside of an eye, the second anatomic space is on top of the eye, and the pressure-sensing element is configured to measure intraocular pressure.

40. The method of claim 37, wherein the first anatomic space is a left atrium of a heart, the second anatomic space is a right atrium of the heart, and the pressure-sensing element is configured to measure pressure in the left atrium.

41. The method of claim 40, wherein the pressure-sensing element and readout electronics are integrated into an Amplatzer device or an inter-atrial shunting device and the implanting the pressure-sensing element and readout electronics further comprises implanting the Amplatzer device or the inter-atrial shunting device.

42. The method of claim 37, wherein the first anatomic space is a first location in a right atrium of a heart, the second anatomic space is a second location in the right atrium of the heart, and the pressure-sensing element is configured to measure pressure in the right atrium.

43. The method of claim 42, wherein the pressure-sensing element and readout electronics are integrated into an Amplatzer device or an inter-atrial shunting device and the implanting the pressure-sensing element and readout electronics further comprises implanting the Amplatzer device or the inter-atrial shunting device.

44. The method of claim 37, wherein the first anatomic space is a first location on a wall of a pulmonary artery, the second anatomic space is a second location on the wall of the pulmonary artery, and the pressure-sensing element is configured to measure pressure in the pulmonary artery.

45. The method of claim 44, wherein the pressure-sensing element and readout electronics are integrated into a stent and the implanting the pressure-sensing element and readout electronics further comprises implanting the stent.

46. The method of claim 37, wherein the first anatomic space is an aneurysm sac, the second anatomic space is an artery, and the pressure-sensing element is configured to measure pressure in the aneurysm sac.

47. The method of claim 46, wherein the pressure-sensing element and readout electronics are integrated into a stent and the implanting the pressure-sensing element and readout electronics further comprises implanting the stent.

48. The method of claim 37, wherein the first anatomic space is an outer wall of a blood vessel, the second anatomic space is a tissue below a cutis, and the pressure-sensing element is configured to measure pressure in the blood vessel.

49. The method of claim 37, wherein the first anatomic space is an inside of a bladder, the second anatomic space is outside of the bladder, and the pressure-sensing element is configured to measure pressure in the bladder.

50. The method of claim 37, wherein the first anatomic space is an inside a graft, the second anatomic space is outside the graft, and the pressure-sensing element is configured to measure pressure in the graft.

51. The method of claim 37, wherein the implanting the pressure-sensing element and readout electronics further comprises endoscopically implanting the pressure-sensing element and readout electronics under direct vision.
